(19) United States
(12) Patent Application Publication
(10) Pub. No.: US 2023/0061020 A1
(43) Pub. Date: Mar. 2, 2023

(54) SYSTEMS AND METHODS FOR TRANSFORMING AN INTERACTIVE GRAPHICAL USER INTERFACE ACCORDING TO DYNAMICALLY GENERATED DATA STRUCTURES

(71) Applicant: Evernorth Strategic Development, Inc., St. Louis, MO (US)

(72) Inventors: Se W. Kim, Franklin Lakes, NJ (US); Venkata Satya Sundara Ramam Durbha, Parsippany, NJ (US); Pola Rajesh Kumar Pilla, Bengaluru (IN); Viju Paul, Fair Lawn, NJ (US); Richard C. Baudisch, JR., Montvale, NJ (US); Elivett Oliver, Saddle Brook, NJ (US); Ahsan Javeed, Parsippany, NJ (US); Laurie Evanchick, West Milford, NJ (US); Julie Bulthaus, Chesterfield, MO (US); Marie A. Vidaic, Wyckoff, NJ (US); Purna Dhru, Old Greenwich, CT (US)

(21) Appl. No.: 17/412,888

(22) Filed: Aug. 26, 2021

(51) Int. Cl. G06F 9/451; G06F 3/0481; G06F 3/0482; G06F 16/25; G06F 16/242

(52) U.S. Cl. CPC G06F 9/451; G06F 3/04812; G06F 3/0482; G06F 16/258; G06F 16/252; G06F 16/2428

(57) ABSTRACT

A system for automatically transforming a graphical user interface including a first data server containing a first raw data structure, a second data server containing a second raw data structure, a storage device containing a third raw data structure, and an interactive graphical user interface generation server including a processor and a non-volatile storage device. The graphical user interface generation server is configured to retrieve the first raw data structure and the second raw data structure, and generate a first processed data structure. The graphical user interface generation server is configured to retrieve the third raw data structure and generate a second processed data structure. The graphical user interface generation server is configured to transform a plurality of selectable outlines displayed on a graphical user interface in response a user selecting the first selectable element and/or the second selectable element.

### FIELD

The present disclosure relates to user interface adaptation and, more particularly, to dynamically transforming an interactive graphical user interface according to automatically generated data structures.

### BACKGROUND

Currently, entities, such as high-volume pharmacies, offer online drug management programs, and often store large quantities of information about pharmacy members. These data about pharmacy members are often stored in raw, unsorted, and unfiltered machine-readable data structures not readily comprehensible to a human reader. Separately, open-source Internet databases and public domain government databases also store a wealth of demographic and public health data. Similarly, these vast quantities of data are also often stored in raw, unsorted, and unfiltered machine-readable data structures not readily comprehensible to the human reader.

Often, the aggregated data structures may contain life-saving insights or, at the very least, information to help individuals and corporations understand the public health risks for a given population of pharmacy members within any given geographic region. However, given the machine-readable nature of these raw data structures, they are often of little use to data scientists and analysts without further processing by machine and presentation in a graphical user interface. Accordingly, there exists a need for a system capable of automatically processing these data structures and dynamically generating interactive graphical user interfaces based on the processed data structures.

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

A system for automatically transforming a graphical user interface according to dynamically generated data structures is presented. The system may include a first data server, a second data server, a storage device, and an interactive graphical user interface generation server. The first data server may include a first raw data structure. The second data server may include a second raw data structure. The storage device may include a third raw data structure. The interactive graphical user interface generation server may include a processor and a non-volatile storage device. The non-volatile storage device may include a data access and analysis module and an interactive graphical user interface generation module.

The processor and the data access and analysis module may be configured to retrieve the first raw data structure from the first data server, retrieve the second raw data structure from the second data server, and generate a first processed data structure from the first raw data structure and the second raw data structure. The processor and the data access and analysis module may be further configured to retrieve the third raw data structure from the third data server, generate a second processed data structure from the third raw data structure, and generate a third processed data structure from the first processed data structure and the second processed data structure.

The processor and the interactive graphical user interface generation server may be configured to transform the graphical user interface by displaying a first selectable element, a second selectable element, and a plurality of selectable outlines according to the third processed data structure. In response to a user selecting the first selectable element, the processor and the interactive graphical user interface generation server may automatically transform a color of each of the plurality of selectable outlines according to the third processed data structure. In response to the user selecting the second selectable element, the processor and the interactive graphical user interface generation server may automatically transform a saturation level of each of the plurality of selectable outlines according to the third processed data structure.

In other features, the third processed data structure may include a plurality of metrics corresponding to a plurality of geographical regions. In other features, each outline of the plurality of selectable outlines may correspond to a geographical region of the plurality of geographical regions. In other features, the third processed data structure may include a plurality of case rate metrics. Each case rate metric may be associated with a corresponding geographical region of the plurality of geographical regions. In other features, the processor and the interactive graphical user interface generation server may be configured to determine whether the corresponding case rate metric of each selectable outline exceeds a first threshold. In response to determining that the corresponding case rate metric of the selectable outline exceeds the first threshold, the processor and the interactive graphical user interface generation server may transform the selectable outline into a first color.

In other features, the processor and the interactive graphical user interface may be configured to determine whether the corresponding case rate metric of each selectable outline exceeds a second threshold. In response to determining that the corresponding case rate metric of the selectable outline exceeds the second threshold, the processor and the interactive graphical user interface generation server may be configured to transform the selectable outline into a second color. In other features, in response to determining that the corresponding case rate metric of the selectable outline does not exceed the second threshold, the processor and the interactive graphical user interface generation server may be configured to transform the selectable outline into a third color.

In other features, the third processed data structure may include a plurality of percentage high risk member metrics. Each percentage high risk member metric may be associated with a corresponding region of the plurality of geographic regions. In other features, the processor and the interactive graphical user interface server may be configured to determine whether the corresponding high risk member metric of each selectable outline exceeds a third threshold. In response to determining that the corresponding high risk member metric of the selectable outline exceeds the third threshold, the processor and the interactive graphical user interface generation server may be configured to transform a saturation of the selectable outline into a first saturation level.

In other features, the processor and the interactive graphical user interface generation server may be configured to determine whether the corresponding high risk member metric of each selectable outline exceeds a fourth threshold. In response to determining that the corresponding high risk member metric of the selectable outline exceeds the fourth threshold, the processor and the interactive graphical user interface generation server may be configured to transform the saturation of the selectable outline into a second saturation level. In response to determining that the corresponding high risk member metric of the selectable outline does not exceed the fourth threshold, the processor and the interactive graphical user interface generation server may be configured to transform the saturation of the selectable outline into a third saturation level. The first saturation level may be greater than the second saturation level, and the second saturation level may be greater than the third saturation level.

A method for automatically transforming a graphical user interface according to dynamically generated data structures is presented. The method may include retrieving a first raw data structure from a first data server, retrieving a second raw data structure from a second data server, generating a first processed data structure from the first raw data structure and the second raw data structure, retrieving a third raw data structure from a third data server, generating a second processed data structure from the third raw data structure, generating a third processed data structure from the first processed data structure and the second processed data structure, transforming the graphical user interface by displaying a first selectable element, transforming the graphical user interface by displaying a second selectable element, and transforming the graphical user interface by displaying a plurality of selectable outlines, each selectable outline generated according to the third processed data structure. In response to a user selecting the first selectable element, the method may include automatically transforming a color of each of the plurality of selectable outlines according to the third processed data structure. In response to the user selecting the second selectable element, the method may include automatically transforming a saturation level of each of the plurality of selectable outlines according to the third processed data structure.

In other features, the third processed data structure may include a plurality of metrics corresponding to a plurality of geographical regions. In other features, each outline of the plurality of selectable outlines may correspond to a geographical region of the plurality of geographical regions. In other features, the third processed data structure may include plurality of case rate metrics. Each case rate metric may be associated with a corresponding geographical region of the plurality of geographical regions.

In other features, the method may include determining whether the corresponding case rate metric of each selectable outline exceeds a first threshold. In response to determining the corresponding case rate metric of the selectable outline exceeds the first threshold, the method may include transforming the selectable outline into a first color. In other features, the method may include determining whether the corresponding case rate metric of each selectable outline exceeds a second threshold. In response to determining the corresponding case rate metric of the selectable outline exceeds the second threshold, the method may include transforming the selectable outline into a second color. In other features, in response to determining the corresponding case rate metric of the selectable outline does not exceed the second threshold, the method may include transforming the selectable outline into a third color.

In other features, the third processed data structure may include a plurality of percentage high risk member metrics. Each percentage high risk member metric may be associated with a corresponding region of the plurality of geographic regions. In other features, the method may include determining whether the corresponding high risk member metric of each selectable outline exceeds a third threshold. In response to determining the corresponding high risk member metric of the selectable outline exceeds the third threshold, the method may include transforming a saturation of the selectable outline into a first saturation level.

In other features, the method may include determining whether the corresponding high risk member metric of each selectable outline exceeds a fourth threshold. In response to determining the corresponding high risk member metric of the selectable outline exceeds the fourth threshold, the method may include transforming a saturation of the selectable outline into a second saturation level. In response to determining the corresponding high risk member metric of the selectable outline does not exceed the fourth threshold, the method may include transforming a saturation of the selectable outline into a third saturation level. The first saturation level may be greater than the second saturation level, and the second saturation level may be greater than the third saturation level.

Further areas of applicability of the present disclosure will become apparent from the detailed description, the claims, and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings.

FIG. 1 is a functional block diagram of an example system including a high-volume pharmacy.

FIG. 2 is a functional block diagram of an example pharmacy fulfillment device, which may be deployed within the system of FIG. 1.

FIG. 3 is a functional block diagram of an example order processing device, which may be deployed within the system of FIG. 1.

FIG. 4 is a functional block diagram of an example interactive graphical user interface generation server.

FIG. 5 is a flowchart of an example process according to principles of the present disclosure.

FIG. 6 is a flowchart of an example process for generating a data structure from raw data retrieved from public data servers.

FIG. 7 is a flowchart of an example process calculating metrics from data extracted from raw data structures.

FIG. 8 is a flowchart of an example process for assembling a refined data structure from data extracted from raw data structures.

FIG. 9 is a flowchart of an example process for generating a refined data structure from raw data retrieved from public data servers.

FIG. 10 is a flowchart of an example process for automatically generating a parsed data structure from refined data structures.

FIG. 11 is a flowchart of an example process for automatically generating an interactive user interface from parsed data structures.

FIG. 12 is a flowchart of an example process for automatically transforming graphical user interface elements according to parsed data structures.

FIG. 13 is a flowchart of an example process for automatically transforming graphical user interface elements according to parsed data structures.

FIG. 14 is a flowchart of an example process for automatically transforming graphical user interface elements according to parsed data structures.

In the drawings, reference numbers may be reused to identify similar and/or identical elements.

### DETAILED DESCRIPTION

### INTRODUCTION

Adapting user interfaces based on automatically generated data structures retrieved from public data servers automatically provides personalized, interactive, and dynamically-generated graphical user interfaces directly to a user. The dynamically-generated graphical user interfaces may parse large volumes of raw data, and use the parsed and restructured data structures to visually transform pixels on a display based on dynamic inputs from the user, presenting the often-incomprehensible raw data in an ergonomic visual manner capable of being understood by a human.

In various implementations, the interactive dynamically-generated graphical user interfaces may be automatically generated using parsed data structures and in response to inputs from the user. In various implementations, the parsed data structures may be automatically generated from a first raw data structure retrieved from a first public data server and a second raw data structure retrieved from a second public data server. Once the first and second raw data structures are obtained, they may be analyzed and joined into the parsed data structure. In various implementations, the interactive dynamically-generated graphical user interfaces may be automatically generated based on relevant data contained within the parsed data structure as the user selects parts of the user interface, dramatically improving the overall ergonomics for the user, analyst, and/or support representative.

### HIGH-VOLUME PHARMACY

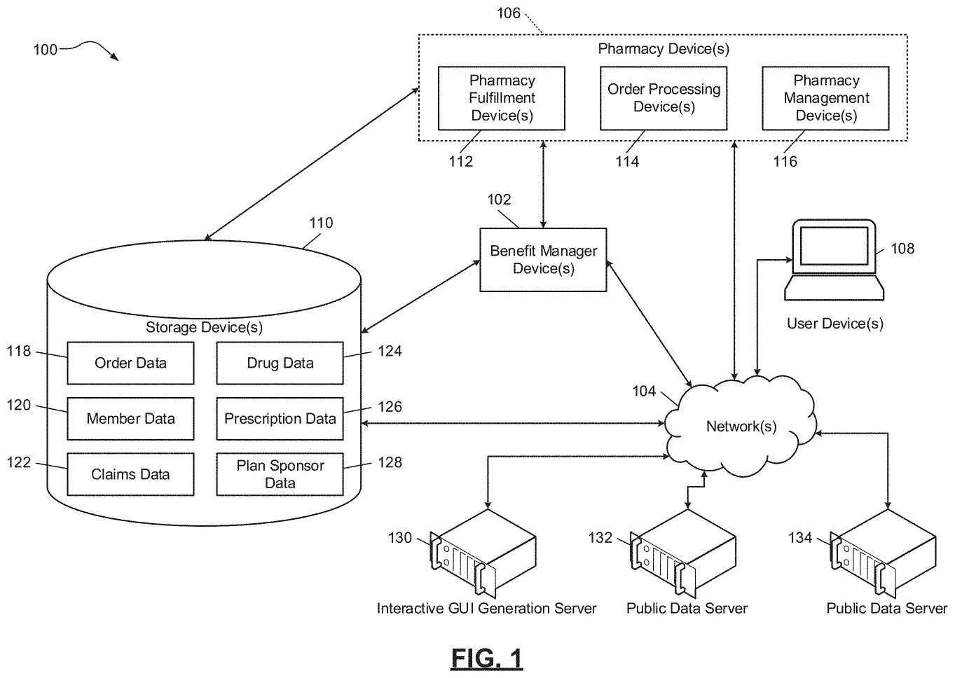

FIG. 1 is a block diagram of an example implementation of a system 100 for a high-volume pharmacy. While the system 100 is generally described as being deployed in a high-volume pharmacy or a fulfillment center (for example, a mail order pharmacy, a direct delivery pharmacy, etc.), the system 100 and/or components of the system 100 may otherwise be deployed (for example, in a lower-volume pharmacy, etc.). A high-volume pharmacy may be a pharmacy that is capable of filling at least some prescriptions mechanically. The system 100 may include a benefit manager device 102 and a pharmacy device 106 in communication with each other directly and/or over a network 104.

The system 100 may also include one or more user device(s) 108. A user, such as a pharmacist, patient, data analyst, health plan administrator, etc., may access the benefit manager device 102 or the pharmacy device 106 using the user device 108. The user device 108 may be a desktop computer, a laptop computer, a tablet, a smartphone, etc.

The benefit manager device 102 is a device operated by an entity that is at least partially responsible for creation and/or management of the pharmacy or drug benefit. While the entity operating the benefit manager device 102 is typically a pharmacy benefit manager (PBM), other entities may operate the benefit manager device 102 on behalf of themselves or other entities (such as PBMs). For example, the benefit manager device 102 may be operated by a health plan, a retail pharmacy chain, a drug wholesaler, a data analytics or other type of software-related company, etc. In some implementations, a PBM that provides the pharmacy benefit may provide one or more additional benefits including a medical or health benefit, a dental benefit, a vision benefit, a wellness benefit, a radiology benefit, a pet care benefit, an insurance benefit, a long term care benefit, a nursing home benefit, etc. The PBM may, in addition to its PBM operations, operate one or more pharmacies. The pharmacies may be retail pharmacies, mail order pharmacies, etc.

Some of the operations of the PBM that operates the benefit manager device 102 may include the following activities and processes. A member (or a person on behalf of the member) of a pharmacy benefit plan may obtain a prescription drug at a retail pharmacy location (e.g., a location of a physical store) from a pharmacist or a pharmacist technician. The member may also obtain the prescription drug through mail order drug delivery from a mail order pharmacy location, such as the system 100. In some implementations, the member may obtain the prescription drug directly or indirectly through the use of a machine, such as a kiosk, a vending unit, a mobile electronic device, or a different type of mechanical device, electrical device, electronic communication device, and/or computing device. Such a machine may be filled with the prescription drug in prescription packaging, which may include multiple prescription components, by the system 100. The pharmacy benefit plan is administered by or through the benefit manager device 102.

The member may have a copayment for the prescription drug that reflects an amount of money that the member is responsible to pay the pharmacy for the prescription drug. The money paid by the member to the pharmacy may come from, as examples, personal funds of the member, a health savings account (HSA) of the member or the member's family, a health reimbursement arrangement (HRA) of the member or the member's family, or a flexible spending account (FSA) of the member or the member's family. In some instances, an employer of the member may directly or indirectly fund or reimburse the member for the copayments.

The amount of the copayment required by the member may vary across different pharmacy benefit plans having different plan sponsors or clients and/or for different prescription drugs. The member's copayment may be a flat copayment (in one example, $10), coinsurance (in one example, 10%), and/or a deductible (for example, responsibility for the first $500 of annual prescription drug expense, etc.) for certain prescription drugs, certain types and/or classes of prescription drugs, and/or all prescription drugs. The copayment may be stored in a storage device 110 or determined by the benefit manager device 102.

In some instances, the member may not pay the copayment or may only pay a portion of the copayment for the prescription drug. For example, if a usual and customary cost for a generic version of a prescription drug is $4, and the member's flat copayment is $20 for the prescription drug, the member may only need to pay $4 to receive the prescription drug. In another example involving a worker's compensation claim, no copayment may be due by the member for the prescription drug.

In addition, copayments may also vary based on different delivery channels for the prescription drug. For example, the copayment for receiving the prescription drug from a mail order pharmacy location may be less than the copayment for receiving the prescription drug from a retail pharmacy location.

In conjunction with receiving a copayment (if any) from the member and dispensing the prescription drug to the member, the pharmacy submits a claim to the PBM for the prescription drug. After receiving the claim, the PBM (such as by using the benefit manager device 102) may perform certain adjudication operations including verifying eligibility for the member, identifying/reviewing an applicable formulary for the member to determine any appropriate copayment, coinsurance, and deductible for the prescription drug, and performing a drug utilization review (DUR) for the member. Further, the PBM may provide a response to the pharmacy (for example, the pharmacy system 100) following performance of at least some of the aforementioned operations.

As part of the adjudication, a plan sponsor (or the PBM on behalf of the plan sponsor) ultimately reimburses the pharmacy for filling the prescription drug when the prescription drug was successfully adjudicated. The aforementioned adjudication operations generally occur before the copayment is received and the prescription drug is dispensed. However in some instances, these operations may occur simultaneously, substantially simultaneously, or in a different order. In addition, more or fewer adjudication operations may be performed as at least part of the adjudication process.

The amount of reimbursement paid to the pharmacy by a plan sponsor and/or money paid by the member may be determined at least partially based on types of pharmacy networks in which the pharmacy is included. In some implementations, the amount may also be determined based on other factors. For example, if the member pays the pharmacy for the prescription drug without using the prescription or drug benefit provided by the PBM, the amount of money paid by the member may be higher than when the member uses the prescription or drug benefit. In some implementations, the amount of money received by the pharmacy for dispensing the prescription drug and for the prescription drug itself may be higher than when the member uses the prescription or drug benefit. Some or all of the foregoing operations may be performed by executing instructions stored in the benefit manager device 102 and/or an additional device.

Examples of the network 104 include a Global System for Mobile Communications (GSM) network, a code division multiple access (CDMA) network, 3rd Generation Partnership Project (3GPP), an Internet Protocol (IP) network, a Wireless Application Protocol (WAP) network, or an IEEE 802.11 standards network, as well as various combinations of the above networks. The network 104 may include an optical network. The network 104 may be a local area network or a global communication network, such as the Internet. In some implementations, the network 104 may include a network dedicated to prescription orders: a prescribing network such as the electronic prescribing network operated by Surescripts of Arlington, Va.

Moreover, although the system shows a single network 104, multiple networks can be used. The multiple networks may communicate in series and/or parallel with each other to link the devices 102-110.

The pharmacy device 106 may be a device associated with a retail pharmacy location (e.g., an exclusive pharmacy location, a grocery store with a retail pharmacy, or a general sales store with a retail pharmacy) or other type of pharmacy location at which a member attempts to obtain a prescription. The pharmacy may use the pharmacy device 106 to submit the claim to the PBM for adjudication.

Additionally, in some implementations, the pharmacy device 106 may enable information exchange between the pharmacy and the PBM. For example, this may allow the sharing of member information such as drug history that may allow the pharmacy to better service a member (for example, by providing more informed therapy consultation and drug interaction information). In some implementations, the benefit manager device 102 may track prescription drug fulfillment and/or other information for users that are not members, or have not identified themselves as members, at the time (or in conjunction with the time) in which they seek to have a prescription filled at a pharmacy.

The pharmacy device 106 may include a pharmacy fulfillment device 112, an order processing device 114, and a pharmacy management device b 116 in communication with each other directly and/or over the network 104. The order processing device 114 may receive information regarding filling prescriptions and may direct an order component to one or more devices of the pharmacy fulfillment device 112 at a pharmacy. The pharmacy fulfillment device 112 may fulfill, dispense, aggregate, and/or pack the order components of the prescription drugs in accordance with one or more prescription orders directed by the order processing device 114.

In general, the order processing device 114 is a device located within or otherwise associated with the pharmacy to enable the pharmacy fulfillment device 112 to fulfill a prescription and dispense prescription drugs. In some implementations, the order processing device 114 may be an external order processing device separate from the pharmacy and in communication with other devices located within the pharmacy.

For example, the external order processing device may communicate with an internal pharmacy order processing device and/or other devices located within the system 100. In some implementations, the external order processing device may have limited functionality (e.g., as operated by a user requesting fulfillment of a prescription drug), while the internal pharmacy order processing device may have greater functionality (e.g., as operated by a pharmacist).

The order processing device 114 may track the prescription order as it is fulfilled by the pharmacy fulfillment device 112. The prescription order may include one or more prescription drugs to be filled by the pharmacy. The order processing device 114 may make pharmacy routing decisions and/or order consolidation decisions for the particular prescription order. The pharmacy routing decisions include what device(s) in the pharmacy are responsible for filling or otherwise handling certain portions of the prescription order. The order consolidation decisions include whether portions of one prescription order or multiple prescription orders should be shipped together for a user or a user family. The order processing device 114 may also track and/or schedule literature or paperwork associated with each prescription order or multiple prescription orders that are being shipped together. In some implementations, the order processing device 114 may operate in combination with the pharmacy management device 116.

The order processing device 114 may include circuitry, a processor, a memory to store data and instructions, and communication functionality. The order processing device 114 is dedicated to performing processes, methods, and/or instructions described in this application. Other types of electronic devices may also be used that are specifically configured to implement the processes, methods, and/or instructions described in further detail below.

In some implementations, at least some functionality of the order processing device 114 may be included in the pharmacy management device 116. The order processing device 114 may be in a client-server relationship with the pharmacy management device 116, in a peer-to-peer relationship with the pharmacy management device 116, or in a different type of relationship with the pharmacy management device 116. The order processing device 114 and/or the pharmacy management device 116 may communicate directly (for example, such as by using a local storage) and/or through the network 104 (such as by using a cloud storage configuration, software as a service, etc.) with the storage device 110.

The storage device 110 may include: non-transitory storage (for example, memory, hard disk, CD-ROM, etc.) in communication with the benefit manager device 102 and/or the pharmacy device 106 directly and/or over the network 104. The non-transitory storage may store order data 118, member data 120, claims data 122, drug data 124, prescription data 126, and/or plan sponsor data 128. Further, the system 100 may include additional devices, which may communicate with each other directly or over the network 104.

The order data 118 may be related to a prescription order. The order data may include type of the prescription drug (for example, drug name and strength) and quantity of the prescription drug. The order data 118 may also include data used for completion of the prescription, such as prescription materials. In general, prescription materials include an electronic copy of information regarding the prescription drug for inclusion with or otherwise in conjunction with the fulfilled prescription. The prescription materials may include electronic information regarding drug interaction warnings, recommended usage, possible side effects, expiration date, date of prescribing, etc. The order data 118 may be used by a high-volume fulfillment center to fulfill a pharmacy order.

In some implementations, the order data 118 includes verification information associated with fulfillment of the prescription in the pharmacy. For example, the order data 118 may include videos and/or images taken of (i) the prescription drug prior to dispensing, during dispensing, and/or after dispensing, (ii) the prescription container (for example, a prescription container and sealing lid, prescription packaging, etc.) used to contain the prescription drug prior to dispensing, during dispensing, and/or after dispensing, (iii) the packaging and/or packaging materials used to ship or otherwise deliver the prescription drug prior to dispensing, during dispensing, and/or after dispensing, and/or (iv) the fulfillment process within the pharmacy. Other types of verification information such as barcode data read from pallets, bins, trays, or carts used to transport prescriptions within the pharmacy may also be stored as order data 118.

The member data 120 includes information regarding the members associated with the PBM. The information stored as member data 120 may include personal information, personal health information, protected health information, etc. Examples of the member data 120 include name, address, telephone number, e-mail address, prescription drug history, etc. The member data 120 may include a plan sponsor identifier that identifies the plan sponsor associated with the member and/or a member identifier that identifies the member to the plan sponsor. The member data 120 may include a member identifier that identifies the plan sponsor associated with the user and/or a user identifier that identifies the user to the plan sponsor. The member data 120 may also include dispensation preferences such as type of label, type of cap, message preferences, language preferences, etc.

The member data 120 may be accessed by various devices in the pharmacy (for example, the high-volume fulfillment center, etc.) to obtain information used for fulfillment and shipping of prescription orders. In some implementations, an external order processing device operated by or on behalf of a member may have access to at least a portion of the member data 120 for review, verification, or other purposes.

In some implementations, the member data 120 may include information for persons who are users of the pharmacy but are not members in the pharmacy benefit plan being provided by the PBM. For example, these users may obtain drugs directly from the pharmacy, through a private label service offered by the pharmacy, the high-volume fulfillment center, or otherwise. In general, the terms “member” and “user” may be used interchangeably.

The claims data 122 includes information regarding pharmacy claims adjudicated by the PBM under a drug benefit program provided by the PBM for one or more plan sponsors. In general, the claims data 122 includes an identification of the client that sponsors the drug benefit program under which the claim is made, and/or the member that purchased the prescription drug giving rise to the claim, the prescription drug that was filled by the pharmacy (e.g., the national drug code number, etc.), the dispensing date, generic indicator, generic product identifier (GPI) number, medication class, the cost of the prescription drug provided under the drug benefit program, the copayment/coinsurance amount, rebate information, and/or member eligibility, etc. Additional information may be included.

In some implementations, other types of claims beyond prescription drug claims may be stored in the claims data 122. For example, medical claims, dental claims, wellness claims, or other types of health-care-related claims for members may be stored as a portion of the claims data 122.

In some implementations, the claims data 122 includes claims that identify the members with whom the claims are associated. Additionally or alternatively, the claims data 122 may include claims that have been de-identified (that is, associated with a unique identifier but not with a particular, identifiable member).

The drug data 124 may include drug name (e.g., technical name and/or common name), other names by which the drug is known, active ingredients, an image of the drug (such as in pill form), etc. The drug data 124 may include information associated with a single medication or multiple medications.

The prescription data 126 may include information regarding prescriptions that may be issued by prescribers on behalf of users, who may be members of the pharmacy benefit plan—for example, to be filled by a pharmacy. Examples of the prescription data 126 include user names, medication or treatment (such as lab tests), dosing information, etc. The prescriptions may include electronic prescriptions or paper prescriptions that have been scanned. In some implementations, the dosing information reflects a frequency of use (e.g., once a day, twice a day, before each meal, etc.) and a duration of use (e.g., a few days, a week, a few weeks, a month, etc.).

In some implementations, the order data 118 may be linked to associated member data 120, claims data 122, drug data 124, and/or prescription data 126.

The plan sponsor data 128 includes information regarding the plan sponsors of the PBM. Examples of the plan sponsor data 128 include company name, company address, contact name, contact telephone number, contact e-mail address, etc.

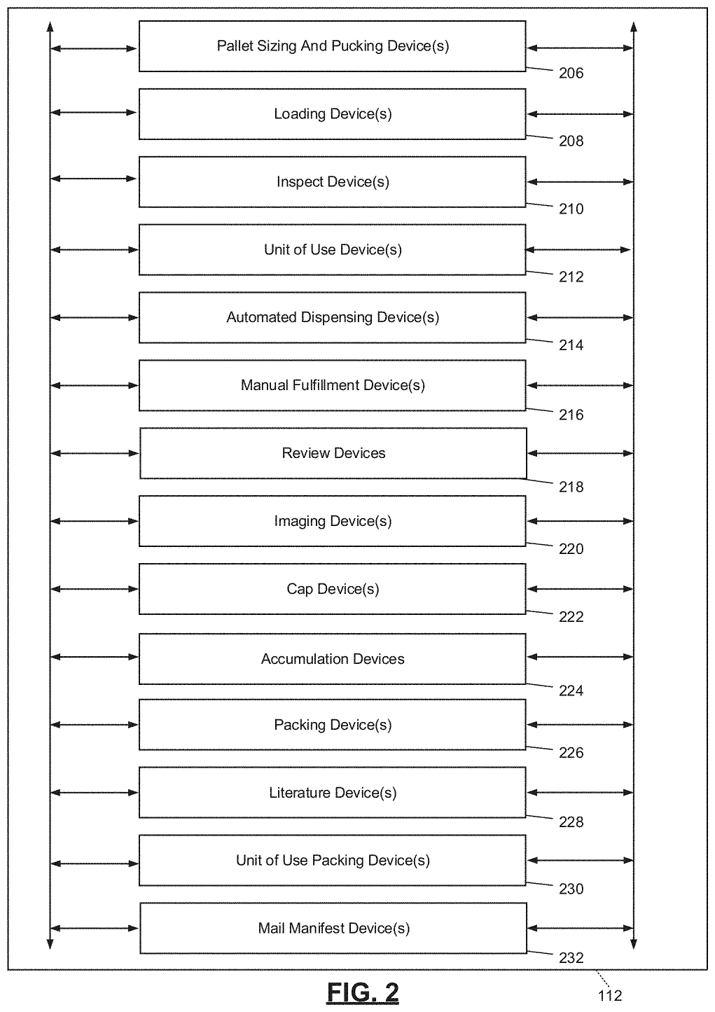

FIG. 2 illustrates the pharmacy fulfillment device 112 according to an example implementation. The pharmacy fulfillment device 112 may be used to process and fulfill prescriptions and prescription orders. After fulfillment, the fulfilled prescriptions are packed for shipping.

The pharmacy fulfillment device 112 may include devices in communication with the benefit manager device 102, the order processing device 114, and/or the storage device 110, directly or over the network 104. Specifically, the pharmacy fulfillment device 112 may include pallet sizing and pucking device(s) 206, loading device(s) 208, inspect device(s) 210, unit of use device(s) 212, automated dispensing device(s) 214, manual fulfillment device(s) 216, review devices 218, imaging device(s) 220, cap device(s) 222, accumulation devices 224, packing device(s) 226, literature device(s) 228, unit of use packing device(s) 230, and mail manifest device(s) 232. Further, the pharmacy fulfillment device 112 may include additional devices, which may communicate with each other directly or over the network 104.

In some implementations, operations performed by one of these devices 206-232 may be performed sequentially, or in parallel with the operations of another device as may be coordinated by the order processing device 114. In some implementations, the order processing device 114 tracks a prescription with the pharmacy based on operations performed by one or more of the devices 206-232.

In some implementations, the pharmacy fulfillment device 112 may transport prescription drug containers, for example, among the devices 206-232 in the high-volume fulfillment center, by use of pallets. The pallet sizing and pucking device 206 may configure pucks in a pallet. A pallet may be a transport structure for a number of prescription containers, and may include a number of cavities. A puck may be placed in one or more than one of the cavities in a pallet by the pallet sizing and pucking device 206. The puck may include a receptacle sized and shaped to receive a prescription container. Such containers may be supported by the pucks during carriage in the pallet. Different pucks may have differently sized and shaped receptacles to accommodate containers of differing sizes, as may be appropriate for different prescriptions.

The arrangement of pucks in a pallet may be determined by the order processing device 114 based on prescriptions that the order processing device 114 decides to launch. The arrangement logic may be implemented directly in the pallet sizing and pucking device 206. Once a prescription is set to be launched, a puck suitable for the appropriate size of container for that prescription may be positioned in a pallet by a robotic arm or pickers. The pallet sizing and pucking device 206 may launch a pallet once pucks have been configured in the pallet.

The loading device 208 may load prescription containers into the pucks on a pallet by a robotic arm, a pick and place mechanism (also referred to as pickers), etc. In various implementations, the loading device 208 has robotic arms or pickers to grasp a prescription container and move it to and from a pallet or a puck. The loading device 208 may also print a label that is appropriate for a container that is to be loaded onto the pallet, and apply the label to the container. The pallet may be located on a conveyor assembly during these operations (e.g., at the high-volume fulfillment center, etc.).

The inspect device 210 may verify that containers in a pallet are correctly labeled and in the correct spot on the pallet. The inspect device 210 may scan the label on one or more containers on the pallet. Labels of containers may be scanned or imaged in full or in part by the inspect device 210. Such imaging may occur after the container has been lifted out of its puck by a robotic arm, picker, etc., or may be otherwise scanned or imaged while retained in the puck. In some implementations, images and/or video captured by the inspect device 210 may be stored in the storage device 110 as order data 118.

The unit of use device 212 may temporarily store, monitor, label, and/or dispense unit of use products. In general, unit of use products are prescription drug products that may be delivered to a user or member without being repackaged at the pharmacy. These products may include pills in a container, pills in a blister pack, inhalers, etc. Prescription drug products dispensed by the unit of use device 212 may be packaged individually or collectively for shipping, or may be shipped in combination with other prescription drugs dispensed by other devices in the high-volume fulfillment center.

At least some of the operations of the devices 206-232 may be directed by the order processing device 114. For example, the manual fulfillment device 216, the review device 218, the automated dispensing device 214, and/or the packing device 226, etc. may receive instructions provided by the order processing device 114.

The automated dispensing device 214 may include one or more devices that dispense prescription drugs or pharmaceuticals into prescription containers in accordance with one or multiple prescription orders. In general, the automated dispensing device 214 may include mechanical and electronic components with, in some implementations, software and/or logic to facilitate pharmaceutical dispensing that would otherwise be performed in a manual fashion by a pharmacist and/or pharmacist technician. For example, the automated dispensing device 214 may include high-volume fillers that fill a number of prescription drug types at a rapid rate and blister pack machines that dispense and pack drugs into a blister pack. Prescription drugs dispensed by the automated dispensing devices 214 may be packaged individually or collectively for shipping, or may be shipped in combination with other prescription drugs dispensed by other devices in the high-volume fulfillment center.

The manual fulfillment device 216 controls how prescriptions are manually fulfilled. For example, the manual fulfillment device 216 may receive or obtain a container and enable fulfillment of the container by a pharmacist or pharmacy technician. In some implementations, the manual fulfillment device 216 provides the filled container to another device in the pharmacy fulfillment devices 112 to be joined with other containers in a prescription order for a user or member.

In general, manual fulfillment may include operations at least partially performed by a pharmacist or a pharmacy technician. For example, a person may retrieve a supply of the prescribed drug, may make an observation, may count out a prescribed quantity of drugs and place them into a prescription container, etc. Some portions of the manual fulfillment process may be automated by use of a machine. For example, counting of capsules, tablets, or pills may be at least partially automated (such as through use of a pill counter). Prescription drugs dispensed by the manual fulfillment device 216 may be packaged individually or collectively for shipping, or may be shipped in combination with other prescription drugs dispensed by other devices in the high-volume fulfillment center.

The review device 218 may process prescription containers to be reviewed by a pharmacist for proper pill count, exception handling, prescription verification, etc. Fulfilled prescriptions may be manually reviewed and/or verified by a pharmacist, as may be required by state or local law. A pharmacist or other licensed pharmacy person who may dispense certain drugs in compliance with local and/or other laws may operate the review device 218 and visually inspect a prescription container that has been filled with a prescription drug. The pharmacist may review, verify, and/or evaluate drug quantity, drug strength, and/or drug interaction concerns, or otherwise perform pharmacist services. The pharmacist may also handle containers which have been flagged as an exception, such as containers with unreadable labels, containers for which the associated prescription order has been canceled, containers with defects, etc. In an example, the manual review can be performed at a manual review station.

The imaging device 220 may image containers once they have been filled with pharmaceuticals. The imaging device 220 may measure a fill height of the pharmaceuticals in the container based on the obtained image to determine if the container is filled to the correct height given the type of pharmaceutical and the number of pills in the prescription. Images of the pills in the container may also be obtained to detect the size of the pills themselves and markings thereon. The images may be transmitted to the order processing device 114 and/or stored in the storage device 110 as part of the order data 118.

The cap device 222 may be used to cap or otherwise seal a prescription container. In some implementations, the cap device 222 may secure a prescription container with a type of cap in accordance with a user preference (e.g., a preference regarding child resistance, etc.), a plan sponsor preference, a prescriber preference, etc. The cap device 222 may also etch a message into the cap, although this process may be performed by a subsequent device in the high-volume fulfillment center.

The accumulation device 224 accumulates various containers of prescription drugs in a prescription order. The accumulation device 224 may accumulate prescription containers from various devices or areas of the pharmacy. For example, the accumulation device 224 may accumulate prescription containers from the unit of use device 212, the automated dispensing device 214, the manual fulfillment device 216, and the review device 218. The accumulation device 224 may be used to group the prescription containers prior to shipment to the member.

The literature device 228 prints, or otherwise generates, literature to include with each prescription drug order. The literature may be printed on multiple sheets of substrates, such as paper, coated paper, printable polymers, or combinations of the above substrates. The literature printed by the literature device 228 may include information required to accompany the prescription drugs included in a prescription order, other information related to prescription drugs in the order, financial information associated with the order (for example, an invoice or an account statement), etc.

In some implementations, the literature device 228 folds or otherwise prepares the literature for inclusion with a prescription drug order (e.g., in a shipping container). In other implementations, the literature device 228 prints the literature and is separate from another device that prepares the printed literature for inclusion with a prescription order.

The packing device 226 packages the prescription order in preparation for shipping the order. The packing device 226 may box, bag, or otherwise package the fulfilled prescription order for delivery. The packing device 226 may further place inserts (e.g., literature or other papers, etc.) into the packaging received from the literature device 228. For example, bulk prescription orders may be shipped in a box, while other prescription orders may be shipped in a bag, which may be a wrap seal bag.

The packing device 226 may label the box or bag with an address and a recipient's name. The label may be printed and affixed to the bag or box, be printed directly onto the bag or box, or otherwise associated with the bag or box. The packing device 226 may sort the box or bag for mailing in an efficient manner (e.g., sort by delivery address, etc.). The packing device 226 may include ice or temperature sensitive elements for prescriptions that are to be kept within a temperature range during shipping (for example, this may be necessary in order to retain efficacy). The ultimate package may then be shipped through postal mail, through a mail order delivery service that ships via ground and/or air (e.g., UPS, FEDEX, or DHL, etc.), through a delivery service, through a locker box at a shipping site (e.g., AMAZON locker or a PO Box, etc.), or otherwise.

The unit of use packing device 230 packages a unit of use prescription order in preparation for shipping the order. The unit of use packing device 230 may include manual scanning of containers to be bagged for shipping to verify each container in the order. In an example implementation, the manual scanning may be performed at a manual scanning station. The pharmacy fulfillment device 112 may also include a mail manifest device 232 to print mailing labels used by the packing device 226 and may print shipping manifests and packing lists.

While the pharmacy fulfillment device 112 in FIG. 2 is shown to include single devices 206-232, multiple devices may be used. When multiple devices are present, the multiple devices may be of the same device type or models, or may be a different device type or model. The types of devices 206-232 shown in FIG. 2 are example devices. In other configurations of the system 100, lesser, additional, or different types of devices may be included.

Moreover, multiple devices may share processing and/or memory resources. The devices 206-232 may be located in the same area or in different locations. For example, the devices 206-232 may be located in a building or set of adjoining buildings. The devices 206-232 may be interconnected (such as by conveyors), networked, and/or otherwise in contact with one another or integrated with one another (e.g., at the high-volume fulfillment center, etc.). In addition, the functionality of a device may be split among a number of discrete devices and/or combined with other devices.

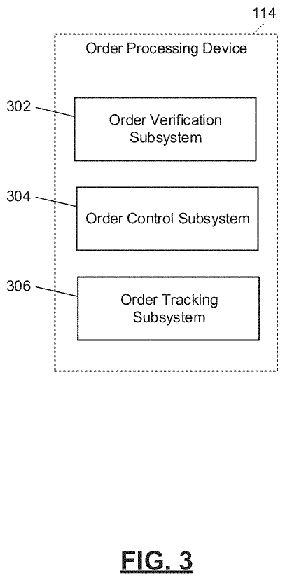

FIG. 3 illustrates the order processing device 114 according to an example implementation. The order processing device 114 may be used by one or more operators to generate prescription orders, make routing decisions, make prescription order consolidation decisions, track literature with the system 100, and/or view order status and other order related information. For example, the prescription order may be comprised of order components.

The order processing device 114 may receive instructions to fulfill an order without operator intervention. An order component may include a prescription drug fulfilled by use of a container through the system 100. The order processing device 114 may include an order verification subsystem 302, an order control subsystem 304, and/or an order tracking subsystem 306. Other subsystems may also be included in the order processing device 114.

The order verification subsystem 302 may communicate with the benefit manager device 102 to verify the eligibility of the member and review the formulary to determine appropriate copayment, coinsurance, and deductible for the prescription drug and/or perform a DUR (drug utilization review). Other communications between the order verification subsystem 302 and the benefit manager device 102 may be performed for a variety of purposes.

The order control subsystem 304 controls various movements of the containers and/or pallets along with various filling functions during their progression through the system 100. In some implementations, the order control subsystem 304 may identify the prescribed drug in one or more than one prescription orders as capable of being fulfilled by the automated dispensing device 214. The order control subsystem 304 may determine which prescriptions are to be launched and may determine that a pallet of automated-fill containers is to be launched.

The order control subsystem 304 may determine that an automated-fill prescription of a specific pharmaceutical is to be launched and may examine a queue of orders awaiting fulfillment for other prescription orders, which will be filled with the same pharmaceutical. The order control subsystem 304 may then launch orders with similar automated-fill pharmaceutical needs together in a pallet to the automated dispensing device 214. As the devices 206-232 may be interconnected by a system of conveyors or other container movement systems, the order control subsystem 304 may control various conveyors: for example, to deliver the pallet from the loading device 208 to the manual fulfillment device 216 from the literature device 228, paperwork as needed to fill the prescription.

The order tracking subsystem 306 may track a prescription order during its progress toward fulfillment. The order tracking subsystem 306 may track, record, and/or update order history, order status, etc. The order tracking subsystem 306 may store data locally (for example, in a memory) or as a portion of the order data 118 stored in the storage device 110.

### INTERACTIVE GRAPHICAL USER INTERFACE GENERATION SERVER AND PUBLIC DATA SERVERS

Referring back to FIG. 1, the system 100 may also include an interactive graphical user interface (GUI) generation server 130. The interactive GUI generation server 130 may communicate with other components of the system 100 directly or through a network, such as network 104. In various implementations, the system 100 may also include one or more data servers, such as public data server 132 and/or public data server 134. In various implementations, public data server 132 and/or public data server 134 may communicate with the interactive GUI generation server 130 through the network 104. In operation, the user device 108 may send a request to the interactive GUI generation server 130. In response to the request from the user device 108, the interactive GUI generation server 130 may generate a first data structure from raw data retrieved from the first public data server 132 and the second public data server 134. In response to the request from the user device 108, the interactive GUI generation server 130 may generate a second data structure from raw data retrieved from storage device 110. The interactive GUI generation server 130 may automatically parse the first data structure and the second data structure to generate a parsed data structure. In various implementations, the interactive GUI generation server 130 may automatically generate an interactive user interface accessible by the user device 108 through network 104 based on the parsed data structure.

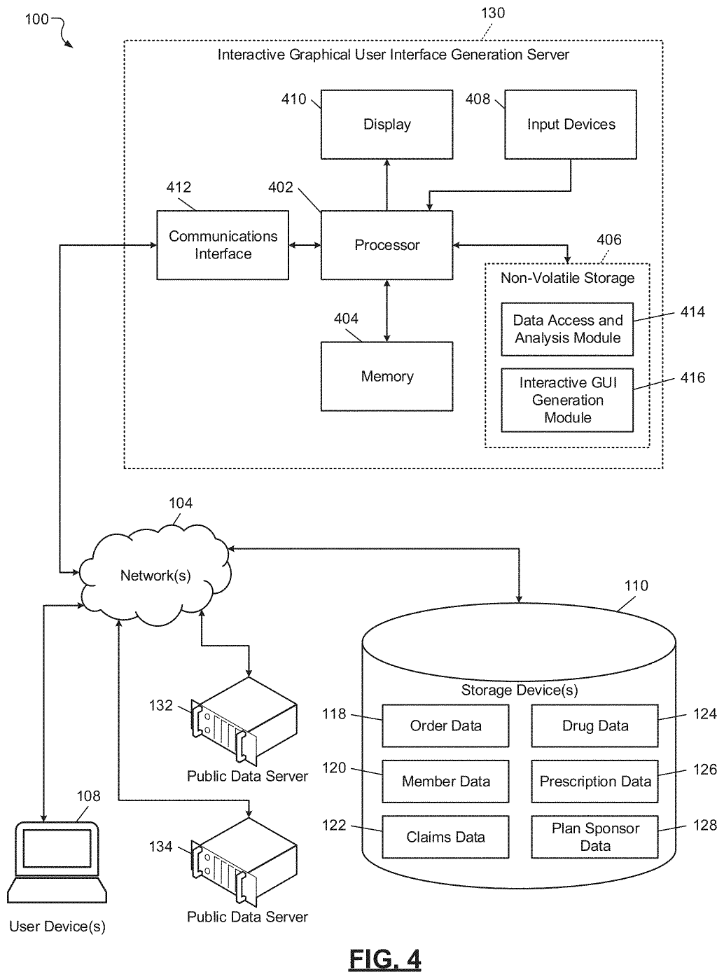

FIG. 4 is a functional block diagram of an example interactive GUI generation server 130. In various implementations, the interactive GUI generation server 130 may include a computer or a microprocessor. For example, interactive GUI generation server 130 may include a processor 402, volatile or non-volatile computer memory 404, such as random-access (RAM), and a non-transitory computer-readable storage medium, such as non-volatile storage 406. In various implementations, the non-volatile storage 406 may include a hard disk drive (HDD), single-level cell (SLC) NAND flash, multi-level cell (MLC) NAND flash, triple-level cell (TLC) NAND flash, quad-level cell (QLC) NAND flash, NOR flash, or any other suitable non-volatile memory or non-volatile storage medium accessible by the processor 402. The interactive GUI generation server 130 may also include one or more input devices, such as input device 408, and one or more output devices, such as display 410. In various implementations, display 410 may be a touchscreen, and may also serve as an input device. In various implementations, the example interactive GUI generation server 130 may also include a transceiver, such as communications interface 412. In various implementations, the memory 404, non-volatile storage 406, input device 408, display 410, and/or communications interface 412 may be operatively coupled to the processor 402 and/or each other.

As illustrated in the example of FIG. 4, the processor 402 may communicate with the network 104 through communications interface 412. In various implementations, processor 402 may access storage device 110, public data server 132, and/or public data server 134 through the communications interface 412 and network 104, or directly through the communications interface 412. In various implementations, the non-volatile storage 406 may include a module for retrieving and parsing data accessed from storage device 110, public data server 132, and/or public data server 134, such as data access and analysis module 414. In various implementations, the non-volatile storage 406 may include a module for dynamically generating an interactive GUI using parsed data structures generated by the data access and analysis module 414, such as interactive GUI generation module 416. In various implementations, user device 108 may access the dynamically generated interactive GUI generated by the interactive GUI generation module 416 through network 104 and communications interface 412, or directly through communications interface 412.

### DATA ACCESS AND ANALYSIS MODULE AND INTERACTIVE GRAPHICAL USER INTERFACE GENERATION MODULE

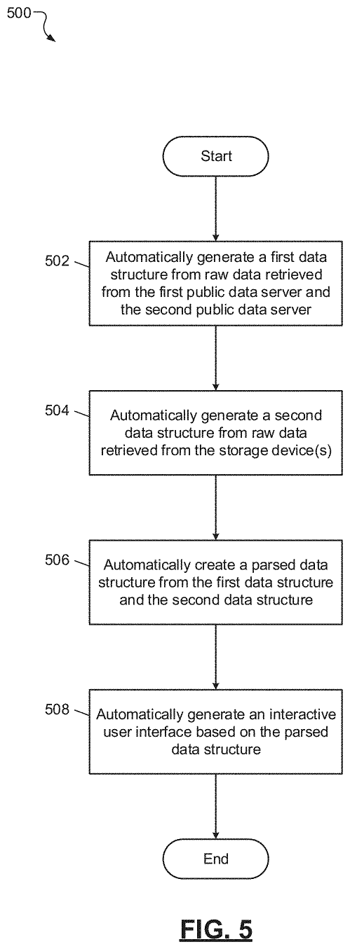

FIG. 5 is a flowchart of an example process 500 which may be performed by the processor 402 and the data access and analysis module 414 and/or the interactive GUI generation module 416 of FIG. 4. Control, such as processor 402, may begin in response to a request received from the user device 108. At 502, the processor 402 and/or the data access and analysis module 414 may automatically generate a first data structure from raw data retrieved from a first public data server, such as public data server 132, and a second public data server, such as public data server 134. Control proceeds to 504. At 504, the processor 402 and/or the data access and analysis module 414 may automatically generate a second data structure from raw data retrieved from the storage device 110. Control proceeds to 506. At 506, the processor 402 and/or data access and analysis module 414 may automatically create a parsed data structure from the first data structure generated at 502 and the second data structure generated at 504. Control proceeds to 508. At 508, the processor 402 and/or the interactive GUI generation module 416 may automatically generate an interactive user interface based on the parsed data structure generated at 506. The user device 108 may access and interact with the generated interactive user interface.

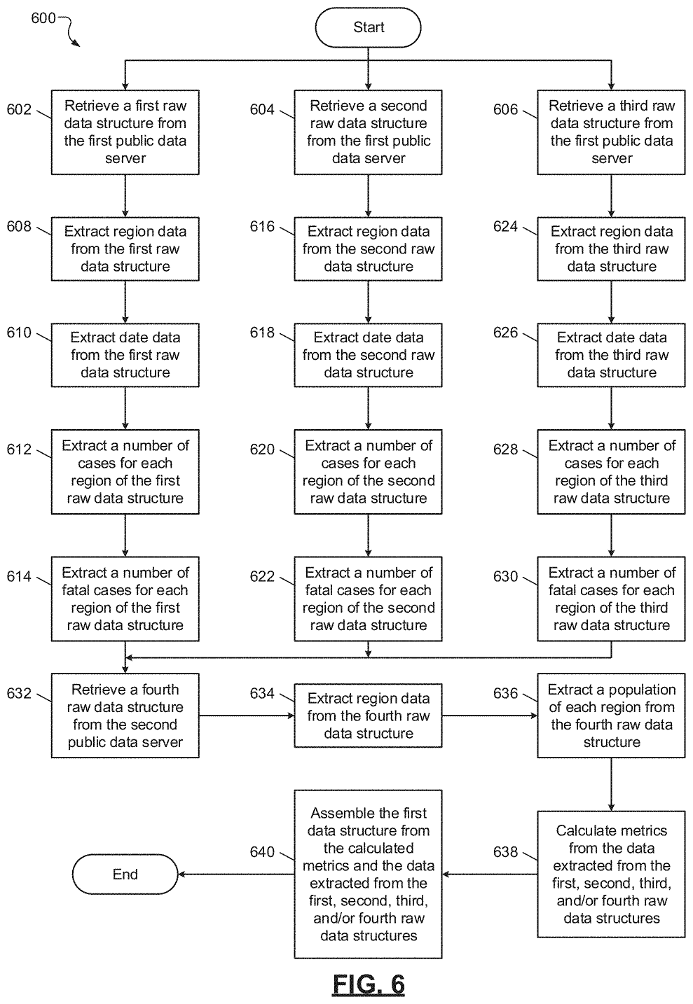

FIG. 6 is a flowchart of an example process 600 which may be performed by the processor 402 and/or the data access and analysis module 414 at step 502 of process 500 of FIG. 5. Control begins at 602, 604, and/or 606. In various implementations, control may execute 602, 604, and/or 606 simultaneously. In various implementations, control may execute 602, 604, and/or 606 in any logical sequential order. At 602, the processor 402 and/or the data access and analysis module 414 retrieves a first raw data structure from the first public data server 132. In various implementations, the first raw data structure may be one or more comma-separated values (CSV) files. In various implementations, the first raw data structure may contain the fields described by Table 1 below:

TABLE 1

| Field | |
|---|---|
| Name | Description |
| date | This field may describe the date that the row of the first raw data |
| | structure was updated |
| county | This field may describe the county that the cases and deaths |
| | values are for |
| state | This field may describe the state that the county value is within |
| ftps | This field may describe the Federal Information Processing |
| | Standards (FIPS) code promulgated by the National Institute of |
| | Standards and Technology (NIST) describing the county |
| cases | This field may describe the total number of positive coronavirus |
| | disease 2019 (COVID-19) cases reported within the county as of |
| | the date |
| deaths | This field may describe the total number of deaths or fatal cases |
| | from COVID-19 within the county as of the date |

In various implementations, each row of the first raw data structure may be structured as follows (with each related field being separated by a comma): date, county, state, fips, cases, deaths

An example of the first raw data structure showing the total number of COVID-19 cases and fatal cases from Oakland County, Michigan, Collin County, Texas, St. Louis County, Missouri, and Fairfax County, Virginia as of Jun. 27, 2021 is represented below: 2021-06-27, Oakland,Michigan,26125,118669,2442 2021-06-27, Collin, Texas,48085,92783,845 2021-06-27, St. Louis, Missouri,29189,101505,2265 2021-06-27, Fairfax, Virginia,51059,77098,1113

After the first raw data structure has been retrieved at 602, control proceeds to 608. At 608, the processor 402 and/or the data access and analysis module 414 extracts region data from the first raw data structure. For example, the processor 402 and/or the data access and analysis module 414 might extract the county, state, and/or fips field(s). Control proceeds to 610. At 610, the processor 402 and/or the data access and analysis module 414 may extract date data from the first raw data structure. For example, the processor 402 and/or the data access and analysis module 414 may extract the date field. Control proceeds to 614. At 612, the processor 402 and/or the data access and analysis module 414 may extract a number of cases for each region of the first data structure. For example, the processor 402 and/or the data access and analysis module 414 may extract the cases field. Control proceeds to 614. At 614, the processor 402 and/or the data access and analysis module 414 may extract a number of fatal cases for each region of the first data structure. For example, the processor 402 and/or the data access and analysis module 414 may extract the deaths field. Control proceeds to 632.

At 604, the processor 402 and/or the data access and analysis module 414 may retrieve a second raw data structure from the first public data server 132. In various implementations, the second raw data structure may be substantially similar to the first raw data structure, except that the second raw data structure may reflect data from a day before the date reflected in the date field of the first data structure. For example, if the first raw data structure contains data as of Jun. 27, 2021, the second raw data structure may contain data as of Jun. 26, 2021: 2021-06-26, Oakland,Michigan,26125,118669,2442 2021-06-26, Collin,Texas,48085,92778,845 2021-06-26, St. Louis,Missouri,29189,101451,2265 2021-06-26, Fairfax,Virginia,51059,77091,1113

After the second raw data structure has been retrieved at 604, control proceeds to 616. At 616, the processor 402 and/or the data access and analysis module 414 may extract region data (e.g., county, state, and/or fips) from the second raw data structure. Control proceeds to 618. At 618, the processor 402 and/or the data access and analysis module 414 may extract date data (e.g., date) from the second raw data structure. Control proceeds to 620. At 620, the processor 402 and/or the data access and analysis module 414 may extract a number of cases (e.g., cases) for each region of the second raw data structure. Control proceeds to 622. At 622, the processor 402 and/or the data access and analysis module 414 may extract a number of fatal cases (e.g., deaths) for each region of the second raw data structure. Control proceeds to 632.

At 606, the processor 402 and/or the data access and analysis module 414 may retrieve a third data module from the first public data server 132. In various implementations, the third raw data structure may be substantially similar to the first raw data structure, except that the third raw data structure may reflect data from a week before the date reflected in the date field of the first data structure. For example, if the first raw data structure contains data as of Jun. 27, 2021, the third raw data structure may contain data as of Jun. 20, 2021: 2021-06-20, Oakland,Michigan,26125,118538,2426 2021-06-20, Collin,Texas,48085,92550,841 2021-06-20, St. Louis,Missouri,29189,101014,2264 2021-06-20, Fairfax,Virginia,51059,77025,1109

After the third raw data structure has been retrieved at 606, control proceeds to 624. At 624, the processor 402 and/or the data access and analysis module 414 may extract region data (e.g., county, state, and/or fips) from the third raw data structure. Control proceeds to 626. At 626, the processor 402 and/or the data access analysis module 414 may extract date data (e.g., date) from the third raw data structure. Control proceeds to 628. At 628, the processor 402 and/or the data access and analysis module 414 may extract a number of cases (e.g., cases) for each region of the third raw data structure. Control proceeds to 630. At 630, the processor 402 and/or the data access and analysis module 414 may extract a number of fatal cases (e.g., deaths) for each region of the third raw data structure. Control proceeds to 632.

At 632, the processor 402 and/or the data access and analysis module 414 retrieves a fourth raw data structure from the second public data server 134. In various implementations, the fourth raw data structure may be a CSV file, and include a county field, a state field, and a population field reflecting the population of a county. In various implementations, each row of the fourth raw data structure may be structured as follows (with each related field being separated by a comma): county, state, population

An example of the fourth raw data structure showing the populations of Oakland County, Michigan, Collin County, Texas, St. Louis County, Missouri, and Fairfax County, Virginia is represented below: Oakland County, Michigan, 1257584 Collin County, Texas, 1034730 St. Louis County, Missouri, 994205 Fairfax County, Virginia, 1147532

After the fourth raw data structure is retrieved at 632, control proceeds to 634. At 634, the processor 402 and/or the data access and analysis module 414 extracts region data from the fourth raw data structure. For example, the processor 402 and/or the data access and analysis module 414 extracts the county and/or state fields from the fourth raw data structure. Control proceeds to 636. At 636, the processor 402 and/or the data access and analysis module 414 extracts a population of each region from the fourth raw data structure. For example, the processor 402 and/or the data access and analysis module 414 extracts the population field of each region from the fourth raw data structure. Control proceeds to 638. At 638, the processor 402 and/or the data access and analysis module 414 calculates metrics from the data extracted from the first, second, third, and/or fourth raw data structures. Control proceeds to 640. At 640, the processor 402 and/or the data access and analysis module 414 assembles the first data structure from the calculated metrics and the data extracted from the first, second, third, and/or fourth raw data structures.

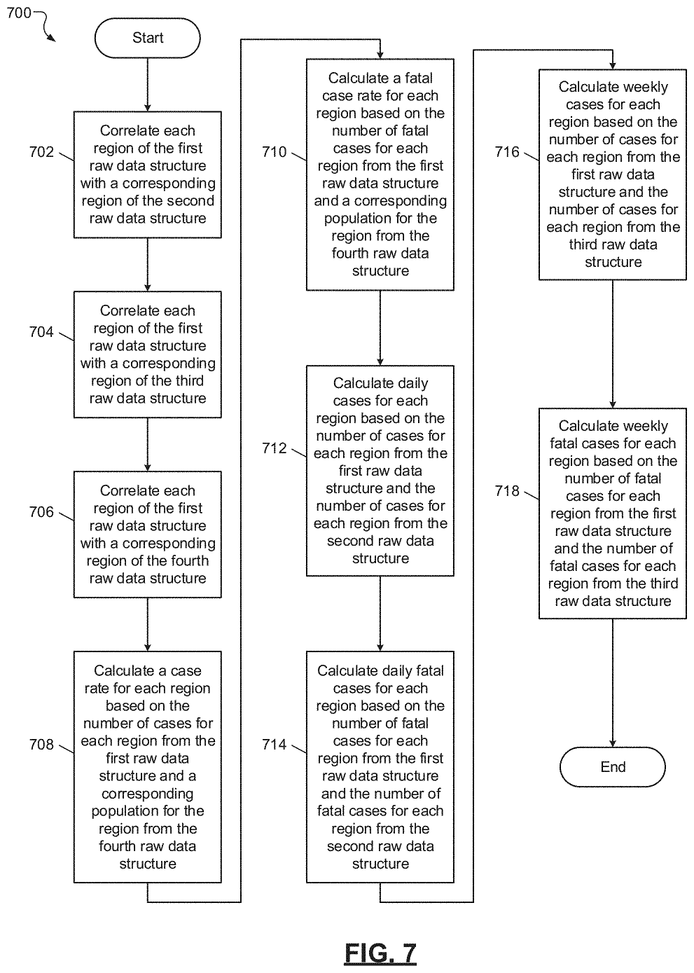

FIG. 7 is a flowchart of an example process 700 which may be performed by the processor 402 and/or the data access and analysis module 414 at step 638 of process 600 of FIG. 6. Control begins at 702. At 702, the processor 402 and/or the data access and analysis module 414 correlates each region of the first raw data structure with a corresponding region of the second raw data structure. For example, the row containing “Oakland,Michigan” of the first raw data structure may be correlated with the row containing “Oakland,Michigan” in the second raw data structure. Control proceeds to 704. At 704, the processor 402 and/or the data access and analysis module 414 correlates each region of the first raw data structure with a corresponding region of the third raw data structure. For example, the row containing “Oakland,Michigan” of the first raw data structure may be correlated with the row containing “Oakland,Michigan” in the third raw data structure. Control proceeds to 706.

At 706, the processor 402 and/or the data access and analysis module 414 correlates each region of the first raw data structure with a corresponding region of the fourth raw data structure. For example, the row containing “Oakland,Michigan” of the first raw data structure may be correlated with the row containing “Oakland County, Michigan” in the fourth raw data structure. Control proceeds to 708. At 708, the processor and/or the data access and analysis module 414 calculates a case rate for each region based on the number of cases for each region from the first raw data structure and a corresponding population for the region from the fourth raw data structure. In various implementations, the case rate may be the number of cases for each 100,000 people within the region. In various implementations, the case rate may be calculated according to Equation (1) below:

$$case rate = \frac{100000 \cdot cases}{population} \qquad ( 1 )$$

For example, the case rate for Oakland County, Michigan may be calculated using the following rows from the first raw data structure and the fourth raw data structure (presented in respective order below): 2021-06-27, Oakland,Michigan,26125,118669,2442 Oakland County, Michigan, 1257584

Thus, the processor 402 and/or the data access and analysis module 414 may calculate the case rate to be about 9,436 cases per 100,000 people for Oakland County, Michigan. The case rate may be calculated for each region within the first raw data structure. Control proceeds to 710.

At 710, the processor 402 and/or the data access and analysis module 414 may calculate a fatal case rate for each region based on the number of fatal cases for each region from the first raw data structure and a corresponding population from the fourth raw data structure. In various implementations, the fatal case rate may be the number of fatal cases for each 100,000 people within the region. In various implementations, the fatal case rate may be calculated according to Equation (2) below:

$$fatal case rate = \frac{100000 \cdot fatal cases}{population} \qquad ( 2 )$$

Thus, the processor 402 and/or the data access and analysis module 414 may calculate the fatal case rate to be about 194 fatal cases per 100,000 people for Oakland County, Michigan. The fatal case rate may be calculated for each region within the first raw data structure. Control proceeds to 712.

At 712, the processor 402 and/or the data access and analysis module 414 may calculate daily cases for each region based on the number of cases for each region from the first raw data structure and the number of cases for each region from the second raw data structure. The daily cases may reflect the number of new cases added to each region over the past day. For example, the case rate for Oakland County, Michigan may be calculated using the following rows from the first raw data structure and the second raw data structure (presented in respective order): 2021-06-27, Oakland,Michigan,26125,118669,2442 2021-06-26, Oakland,Michigan,26125,118669,2442

In various implementations, the daily cases may be calculated according to Equation (3) below:

daily cases=cases_{selected day}-cases_{selected day-1} (3)

Thus, the processor 402 and/or the data access and analysis module 414 may calculate the daily cases to be about 0 cases for Oakland County, Michigan on Jun. 27, 2021. Control proceeds to 714. At 714, the processor 402 and/or the data access and analysis module 414 may calculate daily fatal cases for each region based on the number of fatal cases for each region from the first raw data structure and the number of fatal cases for each region from the second raw data structure. The daily fatal cases may reflect the number of new fatal cases added to each region over the past day. In various implementations, the daily fatal cases for Oakland County, Michigan may be calculated using the rows from the first raw data structure and the second raw data structure previously described with respect to calculating the daily cases. In various implementations, the daily fatal cases may be calculated according to Equation (4) below:

$$\begin{matrix}daily fatal cases \\ = fatal {cases}_{selected day} \\ - fatal {cases}_{selected day - 1}\end{matrix} \qquad ( 4 )$$

Thus, the processor 402 and/or the data access and analysis module 414 may calculate the daily fatal cases to be about 0 cases for Oakland County, Michigan on Jun. 27, 2021. Control proceeds to 716.

At 716, the processor 402 and/or the data access and analysis module 414 may calculate the weekly cases for each region based on the number of cases for each region from the first raw data structure and the number of cases for each region from the third raw data structure. The weekly cases may reflect the number of new cases added to each region over the past week. In various implementations, the weekly cases may reflect the number of new cases added to each region over the past seven days. For example, the case rate for Oakland County, Michigan may be calculated using the following rows from the first raw data structure and the third raw data structure (presented in respective order): 2021-06-27, Oakland,Michigan,26125,118669,2442 2021-06-20, Oakland,Michigan,26125,118538,2426

In various implementations, the weekly cases may be calculated according to Equation (5) below:

weekly cases=cases_{selected day}-cases_{selected day-7} (5)

Thus, the processor 402 and/or the data access and analysis module 414 may calculate the weekly cases to be about 131 cases for Oakland County, Michigan on Jun. 27, 2021. Control proceeds to 718. At 718, the processor 402 and/or the data access and analysis module 414 may calculate the weekly fatal cases for each region based on the number of fatal cases for each region from the first raw data structure and the number of fatal cases for each region from the third raw data structure. The weekly fatal cases may reflect the number of new fatal cases added to each region over the past day. In various implementations, the weekly fatal cases for Oakland County, Michigan may be calculated using the rows from the first raw data structure and the third raw data structure previously described with respect to calculating the weekly cases. In various implementations, the weekly fatal cases may be calculated according to Equation (6) below:

$$\begin{matrix}weekly fatal cases \\ = fatal {cases}_{selected day} \\ - fatal {cases}_{selected day - 7}\end{matrix} \qquad ( 4 )$$

Thus, the processor 402 and/or the data access and analysis module 414 may calculate the weekly fatal cases to be about 16 cases for Oakland County, Michigan on Jun. 27, 2021.

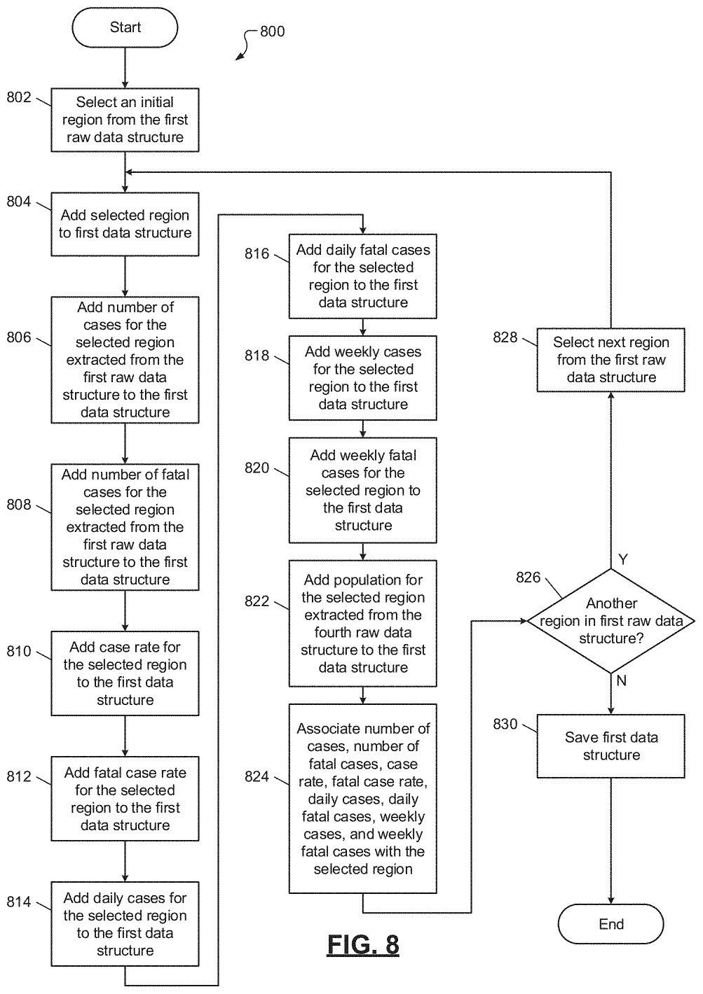

FIG. 8 is a flowchart of an example process 800 which may be performed by the processor 402 and/or the data access and analysis module 414 at step 640 of process 600 of FIG. 6. Control begins at 802. At 802, the processor 402 and/or the data access and analysis module 414 selects an initial region from the first raw data structure. Control proceeds to 804. At 804, the processor 402 and/or the data access and analysis module 414 adds the selected region to the first data structure. Control proceeds to 806. At 806, the processor 402 and/or the data access and analysis module 414 adds the number of cases for the selected region extracted from the first raw data structure to the first data structure. Control proceeds to 808. At 808, the processor 402 and/or the data access and analysis module 414 adds the number of fatal cases for the selected region extracted from the first raw data structure to the first data structure 808. Control proceeds to 810.

At 810, the processor 402 and/or the data access and analysis module 414 adds the case rate for the selected region to the first data structure. Control proceeds to 812. At 812, the processor 402 and/or the data access and analysis module 414 adds the fatal case rate for the selected region to the first data structure. Control proceeds to 814. At 814, the processor 402 and/or the data access and analysis module 414 adds daily cases for the selected region to the first data structure. Control proceeds to 816. At 816, the processor 402 and/or the data access and analysis module 414 adds daily fatal cases for the selected region to the first data structure. Control proceeds to 818. At 818, the processor 402 and/or the data access and analysis module 414 adds weekly cases for the selected region to the first data structure. Control proceeds to 820. At 820, the processor 402 and/or the data access and analysis module 414 adds weekly fatal cases for the selected region to the first data structure. Control proceeds to 822. At 822, the processor 402 and/or the data access and analysis module 414 adds the population for the selected region extracted from the fourth raw data structure to the first data structure. Control proceeds to 824.

At 824, the processor 402 and/or the data access and analysis module 414 associates the number of cases, number of fatal cases, case rate, fatal case rate, daily cases daily fatal cases, weekly cases, and weekly fatal cases with the selected region. Control proceeds to 826. At 826, the processor 402 and/or the data access and analysis module 414 determines whether there is another region in the first raw data structure which has not been parsed and/or added to the first data structure. If at 826, the processor 402 and/or the data access and analysis module 414 determines that the answer is yes, control proceeds to 828. Otherwise, control proceeds to 830. At 828, the processor 402 and/or the data access and analysis module 414 selects the next region from the first raw data structure and proceeds to 804. At 830, the processor 402 and/or the data access and analysis module 414 saves the first data structure. In various implementations, the saved first data structure may include the fields described in Table 2 below:

TABLE 2

| Field | |
|---|---|
| Name | Description |
| Region | This field describes the geographic region that the data and |
| | metrics is associated with |
| Popu- | This field describes the population of the Region as of the |
| lation | Date. |
| Cases | This field describes the total (aggregate) number of positive |
| | cases of COVID-19 reported for the Region up to the Date |
| Fatal | This field describes the total (aggregate) number of fatal |
| Cases | cases or deaths resulting from COVID-19 reported for the |
| | Region up to the Date |
| Case | This field describes the total (aggregate) number of Cases |
| Rate | within the Region for every 100,000 members of the |
| | Population within the Region |
| Fatal | This field describes the total (aggregate) number of Fatal |
| Case | Cases within the Region for every 100,000 members of the |
| Rate | Population within the Region |
| Daily | This field describes the increase in Cases for the Region |
| Cases | over the previous day |
| Daily | This field describes the increase in Fatal Cases for the |
| Fatal | Region over the previous day |
| Cases | |
| Weekly | This field describes the increase in Cases for the Region |
| Cases | over the previous seven days |
| Weekly | This field describes the increase in Fatal Cases for the |
| Fatal | Region over the previous seven days |
| Cases | |

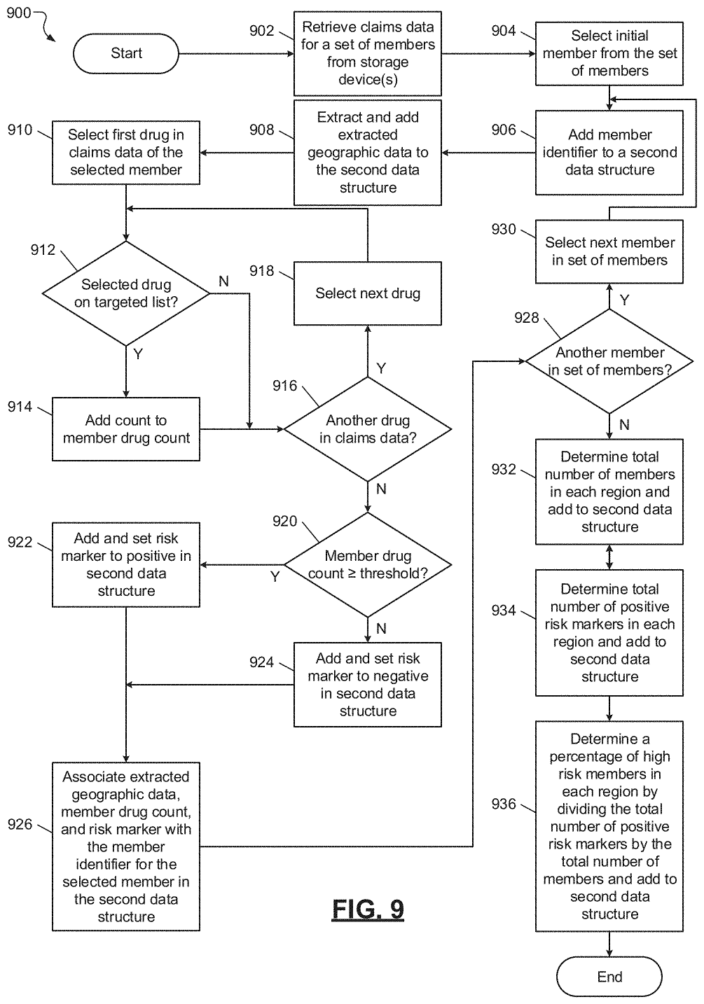

FIG. 9 is a flowchart of an example process 900 which may be performed by the processor 402 and/or the data access and analysis module 414 at step 504 of process 500 of FIG. 5. Control begins at 902. At 902, the processor 402 and/or the data access and analysis module 414 retrieves claims data 122 for a set of members from the storage device 110. Control proceeds to 904. At 904, the processor 402 and/or the data access and analysis module 414 selects an initial member from the set of members. Control proceeds to 906. At 906, the processor 402 and/or the data access and analysis module 414 adds a member identifier for the selected member to a second data structure. In various implementations, the member identifier may be a textual identifier, such as a name, a numerical identifier, such as an identification number assigned by the system 100, a numerical identifier assigned by a governmental agency, or an alphanumerical identifier. The member identifier may serve to link the selected member with data relevant to the selected member in the second data structure. Control proceeds to 908.

At 908, the processor 402 and/or the data access and analysis module 414 extracts geographical data associated with the member from the claims data 122 and adds the extracted geographical data to the second data structure. In various implementations, the geographical data may include a county, state, and/or FIPS code. Control proceeds to 910. At 910, the processor 402 and/or the data access and analysis module 414 selects the first drug present in the claims data 122 of the selected member. Control proceeds to 912. At 912, the processor 402 and/or the data access and analysis module 414 determines whether the selected drug is on a targeted list. In various implementations, the targeted list may contain drugs which may be used to treat medical conditions which place an individual at elevated risk from COVID-19. In various implementations, the targeted list may contain drugs which may be used to treat the conditions and sub-conditions listed in Table 3 below:

TABLE 3

| Condition | Sub-Condition |
|---|---|
| Asthma | Moderate persistent asthma |
| | Severe persistent asthma |
| Chronic lung | Chronic Bronchitis |
| disease | Emphysema |
| | Other chronic obstructive pulmonary disease |
| | Idiopathic pulmonary fibrosis |
| | Cystic fibrosis |
| Diabetes | Type 1 diabetes mellitus |
| | Type 2 diabetes mellitus |
| | Gestational diabetes mellitus |
| Serious | Hypertension or high blood pressure |
| heart | Primary pulmonary hypertension |
| and/or | Other secondary pulmonary hypertension |
| circulatory | Cardiomyopathy |
| conditions | Heart failure |
| | Coronary artery disease (CAD) |
| | Rheumatic heart failure |
| | Hypertensive heart disease with heart failure |
| | Cerebrovascular diseases |
| Chronic | Chronic kidney disease |
| kidney | |
| disease | |
| Severe | Severe obesity (BMI ≥ 30) |
| obesity | |
| Cancer | Cancer treatment |
| Immuno- | Human immunodeficiency virus (HIV) |
| compromised | Transplant |
| | Certain disorders involving the immune |
| | mechanism |
| | Long-term glucocorticosteroid usage (systemic) |
| | Drug-induced immunosuppression (includes |
| | multiple sclerosis drugs) |
| | Drug-induced immunosuppression (includes |
| | drugs used for inflammatory conditions) |
| Hemoglobin | Sickle cell disorders |
| disorders | Thalassemia |
| Liver | Diseases of liver |
| diseases | Acute hepatitis B |
| | Chronic viral hepatitis B with delta-agent |
| | Chronic viral hepatitis B without delta-agent |
| | Unspecified viral hepatitis B |
| | Acute hepatitis C |
| | Chronic viral hepatitis C |
| | Unspecified viral hepatitis C |
| Pregnancy | All trimesters |
| Neurological | ALS |
| diseases | Dementia (includes Alzheimer's Disease) |
| | Down Syndrome |
| Smoking | Smoking and/or vaping |
| People aged | |
| 65 years | |
| and over | |
| Substance use | Use or abuse of prescription and illicit drugs |
| disorder | |

If, at 912, the processor 402 and/or the data access and analysis module 414 determines that the selected drug is on the targeted list, control proceeds to 914. Otherwise, control proceeds to 916.

At 914, the processor 402 and/or the data access and analysis module 414 adds a count to a member drug count. Control proceeds to 916. At 916, the processor 402 and/or the data access and analysis module 414 determines whether another drug is present in the claims data 122 of the selected member. If at 916, the answer is yes, control proceeds to 918, where the processor 402 and/or the data access and analysis module 414 selects the next drug in the claims data 122. If at 916, the answer is no, control proceeds to 920. At 920, the processor 402 and/or the data access and analysis module 414 determines whether the member drug count is greater than or equal to a threshold. In various implementations, the threshold may be selected based on a number of preexisting conditions required to classify the selected member as high risk. In various implementations, the threshold may be three. If at 920, the answer is yes, control proceeds to 922. Otherwise, control proceeds to 924.

At 922, the processor 402 and/or the data access and analysis module 414 adds a risk marker to the second data structure and sets the risk marker to a positive value. Control proceeds to 926. At 924, the processor 402 and/or the data access and analysis module 414 adds the risk marker to the second data structure and sets the risk marker to a negative value. Control proceeds to 926. At 926, the processor 402 and/or the data access and analysis module 414 associates the extracted geographic data, member drug count, and risk marker with the member identifier for the selected member in the second data structure. Control proceeds to 928. At 928, the processor 402 and/or the data access and analysis module 414 determines whether another member is present in the set of members. If at 928, the answer is yes, control proceeds to 930. Otherwise, control proceeds to 932. At 930, the processor 402 and/or the data access and analysis module 414 selects the next member in the set of members. Control returns to 906.

At 932, the processor 402 and/or the data access and analysis module 414 determines a total number of members present in each region and adds the total number of members present in each region to the second data structure. Control proceeds to 934. At 934, the processor 402 and/or the data access and analysis module 414 determines a total number of positive risk markers present in each region and adds the total number of positive risk markers to the second data structure. Control proceeds to 936. At 936, the processor 402 and/or the data access and analysis module 414 determines a percentage of high risk members for each region. In various implementations, the processor 402 and/or the data access and analysis module 414 determines the percentage of high risk members present in each region by dividing the total number of positive risk markers present in each region by the total number of members present in each region. The processor 402 and/or the data access and analysis module 414 adds the percentage of high risk members in each region to the second data structure.

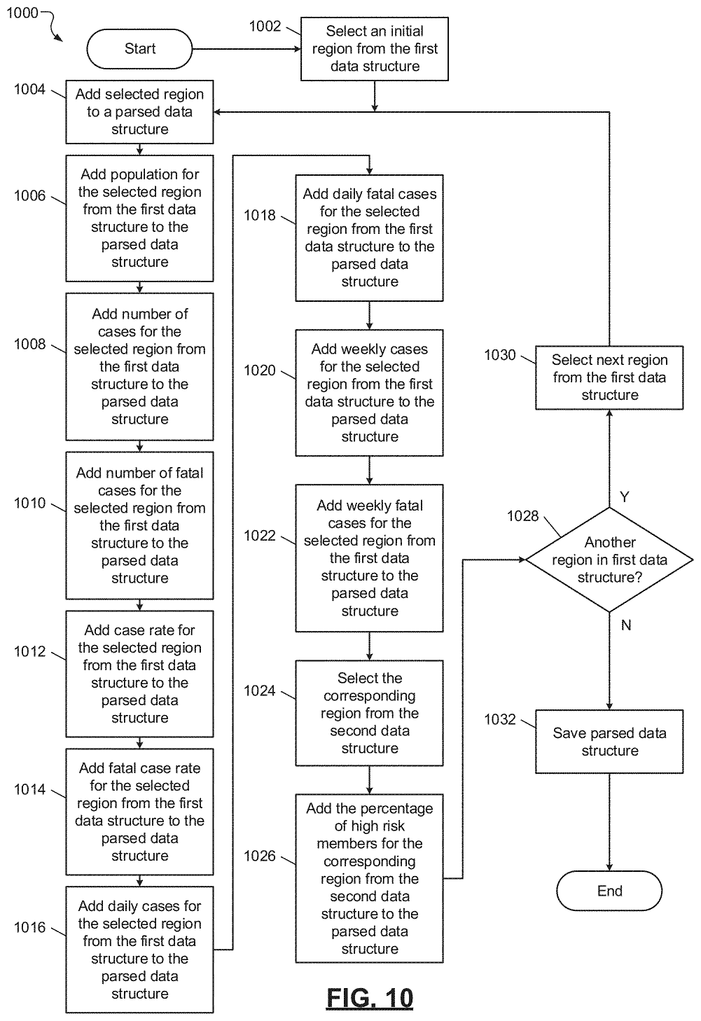

FIG. 10 is a flowchart of an example process 1000 which may be performed by the processor 402 and/or the data access and analysis module 414 at step 506 of process 500 of FIG. 5. Control begins at 1002. At 1002, the processor 402 and/or the data access and analysis module 414 selects an initial region from the first data structure. Control proceeds to 1004. At 1004, the processor 402 and/or the data access and analysis module 414 adds the selected region to a parsed data structure. Control proceeds to 1006. At 1006, the processor 402 and/or the data access and analysis module 414 adds the population for the selected region from the first data structure to the parsed data structure. Control proceeds to 1008. At 1008, the processor 402 and/or the data access and analysis module 414 adds the number of cases for the selected region from the first data structure to the parsed data structure. Control proceeds to 1010. At 1010, the processor 402 and/or the data access and analysis module 414 adds the number of fatal cases for the selected region from the first data structure to the parsed data structure. Control proceeds to 1012.

At 1012, the processor 402 and/or the data access and analysis module 414 adds the case rate for the selected region from the first data structure to the parsed data structure. Control proceeds to 1014. At 1014, the processor 402 and/or the data access and analysis module 414 adds the fatal case rate for the selected region from the first data structure to the parsed data structure. Control proceeds to 1016. At 1016, the processor 402 and/or the data access and analysis module 414 adds daily cases for the selected region from the first data structure to the parsed data structure. Control proceeds to 1018. At 1018, the processor 402 and/or the data access and analysis module 414 adds daily fatal cases for the selected region from the first data structure to the parsed data structure. Control proceeds to 1020. At 1020, the processor 402 and/or the data access and analysis module 414 adds weekly cases for the selected region from the first data structure to the parsed data structure. Control proceeds to 1022. At 1022, the processor 402 and/or the data access and analysis module 414 adds weekly fatal cases for the selected region from the first data structure to the parsed data structure. Control proceeds to 1024.

At 1024, the processor 402 and/or the data access and analysis module 414 selects the corresponding region from the second data structure and proceeds to 1026. At 1026, the processor 402 and/or the data access and analysis module 414 adds the percentage of high risk members for the corresponding region from the second data structure to the parsed data structure. Control proceeds to 1028. At 1028, the processor 402 and/or the data access and analysis module 414 determines whether another region is present in the first data structure. If, at 1028, the answer is yes, control proceeds to 1030. Otherwise, control proceeds to 1032. At 1030, the processor 402 and/or the data access and analysis module 414 selects the next region from the first data structure and proceeds back to 1004. At 1032, the processor 402 and/or the data access and analysis module 414 saves the parsed data structure. In various implementations, the saved parsed data structure may include the fields shown below in Table 4:

TABLE 4

| Field Name | Description |
|---|---|
| Region | This field describes the geographic region that the |
| | data and metrics is associated with |
| Population | This field describes the population of the Region |
| | as of the Date. |
| Cases | This field describes the total (aggregate) number |
| | of positive cases of COVID-19 reported for the |
| | Region up to the Date |
| Fatal Cases | This field describes the total (aggregate) number |
| | of fatal cases or deaths resulting from COVID-19 |
| | reported for the Region up to the Date |
| Case Rate | This field describes the total (aggregate) number |
| | of Cases within the Region for every 100,000 |
| | members of the Population within the Region |
| Fatal Case | This field describes the total (aggregate) number |
| Rate | of Fatal Cases within the Region for every 100,000 |
| | members of the Population within the Region |
| Daily Cases | This field describes the increase in Cases for the |
| | Region over the previous day |
| Daily Fatal | This field describes the increase in Fatal Cases for |
| Cases | the Region over the previous day |
| Weekly | This field describes the increase in Cases for the |
| Cases | Region over the previous seven days |
| Weekly Fatal | This field describes the increase in Fatal Cases for |
| Cases | the Region over the previous seven days |
| Percentage | This field describes the percentage of members in |
| High Risk | the Region which are classified as high risk for |
| Members | COVID-19 |

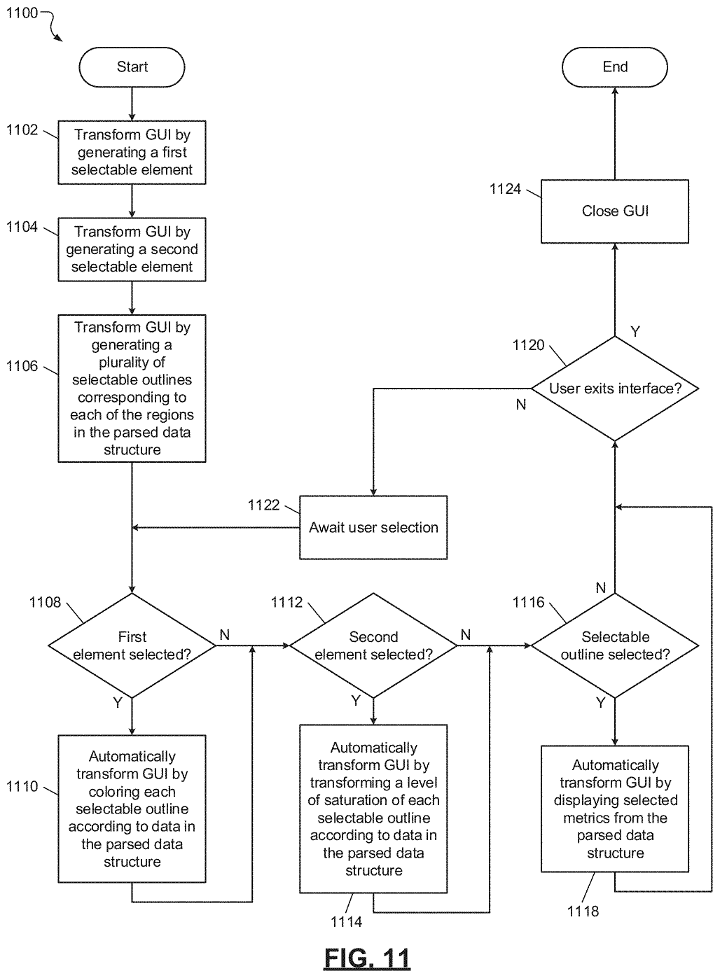

FIG. 11 is a flowchart of an example process 1100 which may be performed by the processor 402 and/or the interactive GUI generation module 416 at step 508 of FIG. 5. Control begins at 1102. At 1102, the processor 402 and/or the interactive GUI generation module 416 transforms a graphical user interface by generating a first selectable element. Control proceeds to 1104. At 1104, the processor 402 and/or the interactive GUI generation module 416 transforms the graphical user interface by generating a second selectable element. Control proceeds to 1106. At 1106, the processor 402 and/or the interactive GUI generation module 416 transforms the graphical user interface by generating a plurality of selectable outlines.

In various implementations, each outline of the plurality of selectable outlines may correspond with one of the regions in the parsed data structure. In various implementations, each outline of the plurality of selectable outlines may have a shape indicative of the physical geographical outline of one of the regions in the parsed data structure. In various implementations, the processor 402 and/or the interactive GUI generation module 416 may generate a selectable outline for each of the regions in the parsed data structure. In various implementations, the processor 402 and/or the interactive GUI generation module 416 may display the plurality of selectable outlines in a layout corresponding to the real-world physical geographical layout of the regions contained in the parsed data structure. In various implementations, each outline of the plurality of selectable outlines may be selected by the user placing a cursor over the outline. In various implementations, each outline of the plurality of selectable outlines may be selected by the user placing a cursor over the outline and clicking the outline. In various implementations, each outline of the plurality of selectable outlines may be selected by the user selecting the outline with a finger or stylus on a touchscreen. Control proceeds to 1108.

At 1108, the processor 402 and/or the interactive GUI generation module 416 determines whether the first element generated at 1102 has been selected. If at 1108, the processor 402 and/or the interactive GUI generation module 416 determines that the user has selected the first element, control proceeds to 1110. Otherwise, control proceeds to 1112. At 1110, the processor 402 and/or the interactive GUI generation module 416 automatically transforms the graphical user interface by coloring each selectable outline according to data in the parsed data structure. Control proceeds to 1112.

At 1112, the processor 402 and/or the interactive GUI generation module 416 determines whether the second element has been selected. If at 1112, the processor 402 and/or the interactive GUI generation module 416 determines that the second element has been selected, control proceeds to 1114. Otherwise, control proceeds to 1116. At 1114, the processor 402 and/or the interactive GUI generation module 416 automatically transforms the graphical user interface by shading each selectable outline according to data in the parsed data structure. Control proceeds to 1116.

At 1116, the processor 402 and/or the interactive GUI generation module 416 determines whether a selectable outline has been selected. If at 1116, the processor 402 and/or the interactive GUI generation module 416 determines that a selectable outline is selected, control proceeds to 1118. Otherwise, control proceeds to 1120. At 1118, the processor 402 and/or the interactive GUI generation module 416 automatically transforms the graphical user interface by displaying selected metrics from the parsed data structure relevant to the region corresponding to the selectable outline. Control proceeds to 1120.

At 1120, the processor 402 and/or the interactive GUI generation module 416 determines whether the user has selected an interface element for exiting the user interface. If at 1120, the answer is no, control proceeds to 1122 and awaits a user selection. Otherwise, control proceeds to 1124 and closes the graphical user interface. After 1122, control proceeds back to 1108.

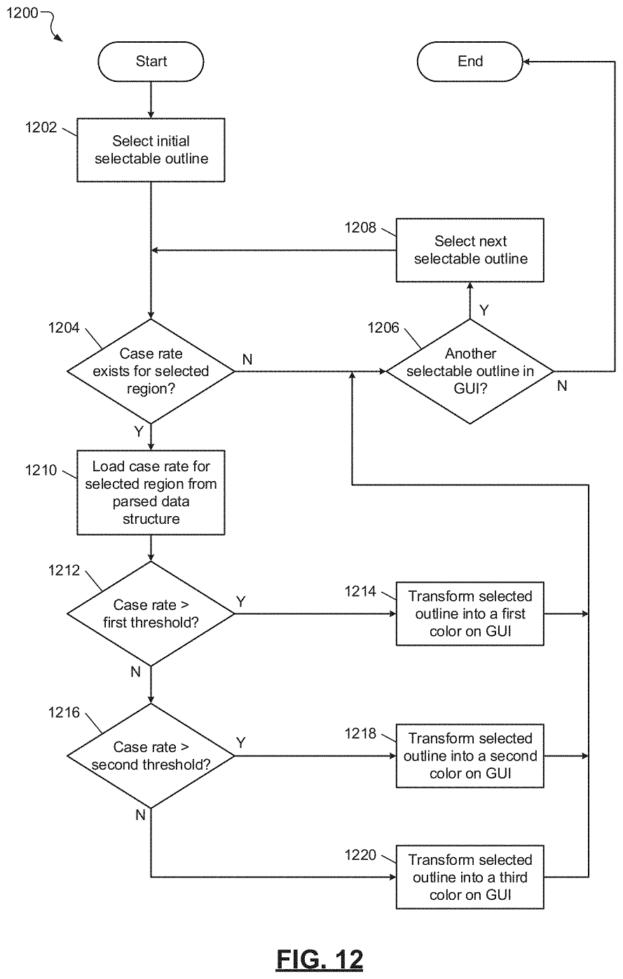

FIG. 12 is a flowchart of an example process 1200 which may be performed by the processor 402 and/or the interactive GUI generation module 416 at step 1110 of process 1100 of FIG. 11. Control begins at 1202. At 1202, the processor 402 and/or the interactive GUI generation module 416 selects the initial selectable outline. Control proceeds to 1204. At 1204, the processor 402 and/or the interactive GUI generation module 416 determines whether a case rate for the region corresponding to the selected selectable outline exists in the parsed data structure. If at 1204, the answer is no, control proceeds to 1206. If at 1204, the answer is yes, control proceeds to 1210.

At 1206, the processor 402 and/or the interactive GUI generation module 416 determines whether another selectable outline exists in the graphical user interface. If at 1206, the answer is yes, control proceeds to 1208. At 1208, the processor 402 and/or the interactive GUI generation module 416 selects the next selectable outline and proceeds back to 1204.

At 1210, the processor 402 and/or the interactive GUI generation module 416 loads the case rate for the region corresponding to the selected selectable outline from the parsed data structure. Control proceeds to 1212. At 1212, the processor 402 and/or the interactive GUI generation module 416 determines whether the loaded case rate exceeds a first threshold. In various implementations, the first threshold may be about 400 cases per 100,000 people. If at 1212, the answer is yes, control proceeds to 1214. Otherwise, control proceeds to 1216. At 1214, the processor 402 and/or the interactive GUI generation module 416 transforms the selected selectable outline into a first color on the graphical user interface. In various implementations, the first color may be red. Control proceeds back to 1206.

At 1216, the processor 402 and/or the interactive GUI generation module 416 determines whether the loaded case rate exceeds a second threshold. In various implementations, the second threshold may be about 100 cases per 100,000 people. If at 1216, the answer is yes, control proceeds to 1218. Otherwise, control proceeds to 1220. At 1218, the processor 402 and/or the interactive GUI generation module 416 transforms the selected selectable outline into a second color on the graphical user interface. In various implementations, the second color may be yellow. Control proceeds back to 1206. At 1220, the processor 402 and/or the interactive GUI generation module 416 transforms the selected selectable outline into a third color on the graphical user interface. In various implementations, the third color may be green. Control proceeds back to 1206.

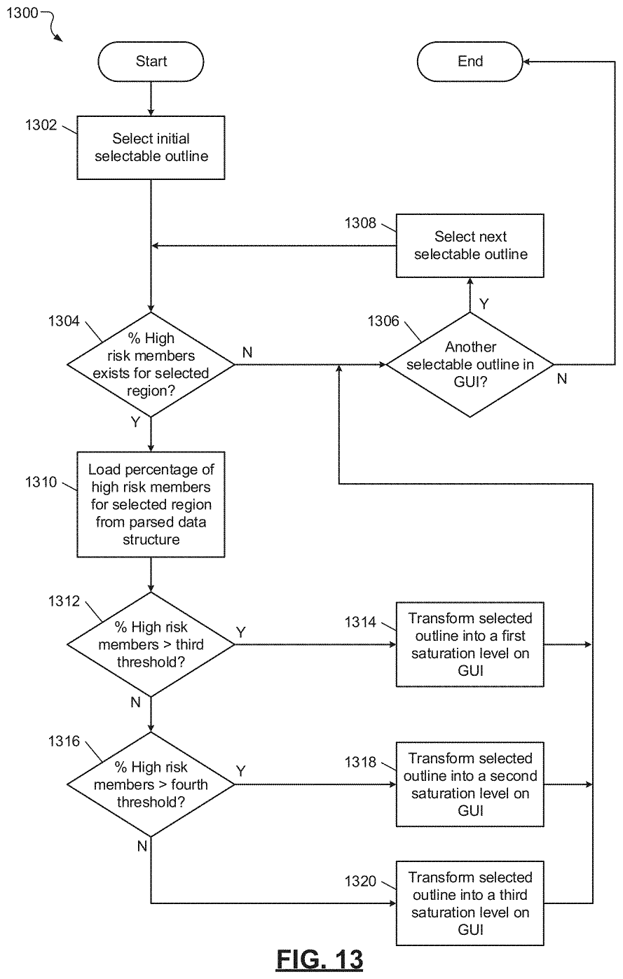

FIG. 13 is a flowchart of an example process 1300 which may be performed by the processor 402 and/or the interactive GUI generation module 416 at step 1114 of process 1100 of FIG. 11. Control begins at 1302. At 1302, the processor 402 and/or the interactive GUI generation module 416 selects the initial selectable outline. Control proceeds to 1304. At 1304, the processor 402 and/or the interactive GUI generation module 416 determines whether the percentage high risk members metric exists for the region corresponding to the selected selectable outline in the parsed data structure. If at 1304, the answer is no, control proceeds to 1306. If at 1304, the answer is yes, control proceeds to 1310.

At 1306, the processor 402 and/or the interactive GUI generation module 416 determines whether another selectable outline exists in the graphical user interface. If at 1306, the answer is yes, control proceeds to 1308. At 1308, the processor 402 and/or the interactive GUI generation module 416 selects the next selectable outline and proceeds back to 1304.

At 1310, the processor 402 and/or the interactive GUI generation module 416 loads the percentage high risk members metric for the region corresponding to the selected selectable outline from the parsed data structure. Control proceeds to 1312. At 1312, the processor 402 and/or the interactive GUI generation module 416 determines whether the loaded percentage high risk members metric exceeds a third threshold. In various implementations, the third threshold may be about 45%. If at 1312, the answer is yes, control proceeds to 1314. Otherwise, control proceeds to 1316. At 1314, the processor 402 and/or the interactive GUI generation module 416 transforms the selected selectable outline into a first level of saturation on the graphical user interface. In various implementations, the first level of saturation may be full saturation. In various implementations, the first level of saturation may be in a range of between about 100% and about 50%. Control proceeds back to 1306.

At 1316, the processor 402 and/or the interactive GUI generation module 416 determines whether the loaded percentage high risk members metric exceeds a fourth threshold. In various implantations, the fourth threshold may be about 30%. If at 1316, the answer is yes, control proceeds to 1318. Otherwise, control proceeds to 1320. At 1318, the processor 402 and/or the interactive GUI generation module 416 transforms the selected selectable outline into a second level of saturation on the graphical user interface. In various implementations, the second level of saturation may be about 75%. In various implementations, the second level of saturation may be in a range of about 75% to about 25%. In various implementations, the second level of saturation may be less than the first level of saturation. Control proceeds back to 1306.

At 1320, the processor 402 and/or the interactive GUI generation module 416 transforms the selected selectable outline into a third level of saturation on the graphical user interface. In various implementations, the third level of saturation may be about 50%. In various implementations, the third level of saturation may be in a range of about 50% to about 0%. In various implementations, the third level of saturation may be less than the second level of saturation. Control proceeds back to 1306.

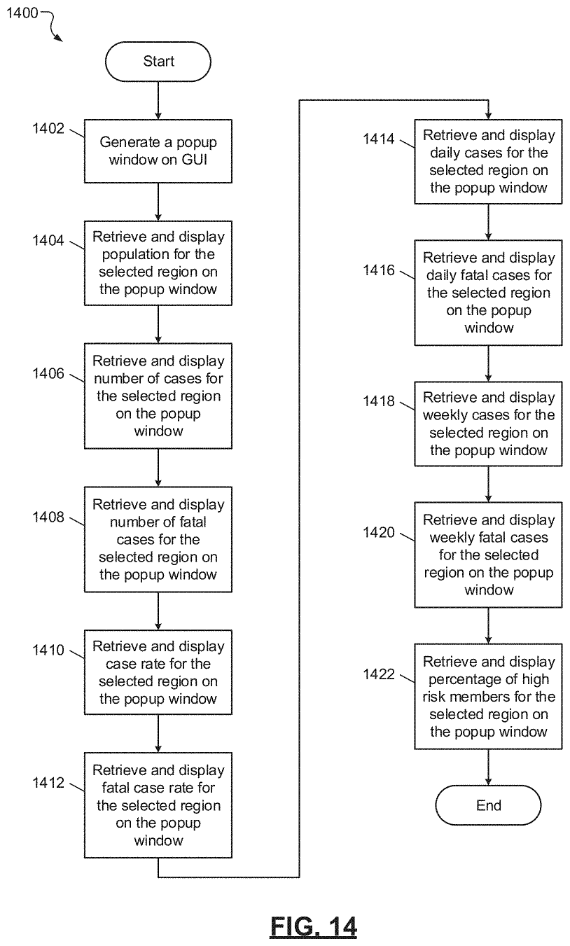

FIG. 14 is a flowchart of an example process 1400 which may be performed by the processor 402 and/or the interactive GUI generation module 416 at step 1118 of process 1100 of FIG. 11. Control begins at 1402. At 1402, the processor 402 and/or the interactive GUI generation module 416 generates a popup window on the graphical user interface. Control proceeds to 1404. At 1404, the processor 402 and/or the interactive GUI generation module 416 retrieves the population metric for the region corresponding to the selected selectable outline from the parsed data structure and displays the population metric in the popup window. Control proceeds to 1406. At 1406, the processor 402 and/or the interactive GUI generation module 416 retrieves the cases metric for the region corresponding to the selected selectable outline from the parsed data structure and displays the cases metric in the popup window. Control proceeds to 1408. At 1408, the processor 402 and/or the interactive GUI generation module 416 retrieves the fatal cases metric for the region corresponding to the selected selectable outline from the parsed data structure and displays the fatal cases metric in the popup window. Control proceeds to 1410.

At 1410, the processor 402 and/or the interactive GUI generation module 416 retrieves the case rate metric for the region corresponding to the selected selectable outline from the parsed data structure and displays the case rate metric in the popup window. Control proceeds to 1412. At 1412, the processor 402 and/or the interactive GUI generation module 416 retrieves the fatal case rate metric for the region corresponding to the selected selectable outline from the parsed data structure and displays the fatal case rate metric in the popup window. Control proceeds to 1414. At 1414, the processor 402 and/or the interactive GUI generation module 416 retrieves the daily cases metric for the region corresponding to the selected selectable outline from the parsed data structure and displays the daily cases metric in the popup window. Control proceeds to 1416. At 1416, the processor 402 and/or the interactive GUI generation module 416 retrieves the daily fatal cases metric for the region corresponding to the selected selectable outline from the parsed data structure and displays the daily fatal cases metric in the popup window. Control proceeds to 1418.

At 1418, the processor 402 and/or the interactive GUI generation module 416 retrieves the weekly cases metric for the region corresponding to the selected selectable outline from the parsed data structure and displays the weekly cases metric in the popup window. Control proceeds to 1420. At 1420, the processor 402 and/or the interactive GUI generation module 416 retrieves the weekly fatal cases metric for the region corresponding to the selected selectable outline from the parsed data structure and displays the weekly fatal cases metric in the popup window. Control proceeds to 1422. At 1422, the processor 402 and/or the interactive GUI generation module 416 retrieves the percentage high risk members metric for the region corresponding to the selected selectable outline from the parsed data structure and displays the percentage high risk members metric in the popup window.

### CONCLUSION

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. In the written description and claims, one or more steps within a method may be executed in a different order (or concurrently) without altering the principles of the present disclosure. Similarly, one or more instructions stored in a non-transitory computer-readable medium may be executed in different order (or concurrently) without altering the principles of the present disclosure. Unless indicated otherwise, numbering or other labeling of instructions or method steps is done for convenient reference, not to indicate a fixed order.

Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements.

The phrase “at least one of A, B, and C” should be construed to mean a logical (A OR B OR C), using a non-exclusive logical OR, and should not be construed to mean “at least one of A, at least one of B, and at least one of C.” The term “set” does not necessarily exclude the empty set—in other words, in some circumstances a “set” may have zero elements. The term “non-empty set” may be used to indicate exclusion of the empty set—in other words, a non-empty set will always have one or more elements. The term “subset” does not necessarily require a proper subset. In other words, a “subset” of a first set may be coextensive with (equal to) the first set. Further, the term “subset” does not necessarily exclude the empty set—in some circumstances a “subset” may have zero elements.

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2020 (also known as the WIFI wireless networking standard) and IEEE Standard 802.3-2015 (also known as the ETHERNET wired networking standard). Examples of a WPAN are IEEE Standard 802.15.4 (including the ZIGBEE standard from the ZigBee Alliance) and, from the Bluetooth Special Interest Group (SIG), the BLUETOOTH wireless networking standard (including Core Specification versions 3.0, 4.0, 4.1, 4.2, 5.0, and 5.1 from the Bluetooth SIG).

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module. For example, the client module may include a native or web application executing on a client device and in network communication with the server module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. Such apparatuses and methods may be described as computerized apparatuses and computerized methods. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, JavaScript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

What is claimed is:

1. A system for automatically transforming a graphical user interface according to dynamically generated data structures, the system comprising:
a first data server configured to store a first raw data structure;
a second data server configured to store a second raw data structure;
a storage device configured to store a third raw data structure; and
an interactive graphical user interface generation server comprising:
a processor, and
a non-volatile storage device comprising instructions that implement:
a data access and analysis module, and
an interactive graphical user interface generation module;
wherein the processor and the data access and analysis module are configured to:
retrieve the first raw data structure from the first data server;
retrieve the second raw data structure from the second data server;
generate a first processed data structure from the first raw data structure and the second raw data structure;
retrieve the third raw data structure from the storage device;
generate a second processed data structure from the third raw data structure; and
generate a third processed data structure from the first processed data structure and the second processed data structure, and
wherein the processor and the interactive graphical user interface generation server are configured to:
transform the graphical user interface by displaying a first selectable element;
transform the graphical user interface by displaying a second selectable element;
transform the graphical user interface by displaying a plurality of selectable outlines according to the third processed data structure;
in response to a user selecting the first selectable element, automatically transform a color of each of the plurality of selectable outlines according to the third processed data structure; and
in response to the user selecting the second selectable element, automatically transform a saturation level of each of the plurality of selectable outlines according to the third processed data structure.

2. The system of claim 1, wherein the third processed data structure comprises a plurality of metrics corresponding to a plurality of geographical regions.

3. The system of claim 2, wherein each outline of the plurality of selectable outlines corresponds to a geographical region of the plurality of geographical regions.

4. The system of claim 3, wherein:
the third processed data structure comprises a plurality of case rate metrics, and
each case rate metric is associated with a corresponding geographical region of the plurality of geographical regions.

5. The system of claim 4, wherein the processor and the interactive graphical user interface generation server are configured to:
determine whether the corresponding case rate metric of each selectable outline exceeds a first threshold; and
in response to determining the corresponding case rate metric of the selectable outline exceeds the first threshold, transform the selectable outline into a first color.

6. The system of claim 5, wherein the processor and the interactive graphical user interface generation server are configured to:
determine whether the corresponding case rate metric of each selectable outline exceeds a second threshold; and
in response to determining the corresponding case rate metric of the selectable outline exceeds the second threshold, transform the selectable outline into a second color.

7. The system of claim 6, wherein the processor and the interactive graphical user interface generation server are configured to:
in response to determining the corresponding case rate metric of the selectable outline does not exceed the second threshold, transform the selectable outline into a third color.

8. The system of claim 3, wherein:
the third processed data structure comprises a plurality of percentage high risk member metrics, and
each percentage high risk member metric is associated with a corresponding region of the plurality of geographic regions.

9. The system of claim 8, wherein the processor and the interactive graphical user interface generation server are configured to:
determine whether the corresponding high risk member metric of each selectable outline exceeds a third threshold; and
in response to determining the corresponding high risk member metric of the selectable outline exceeds the third threshold, transform a saturation of the selectable outline into a first saturation level.

10. The system of claim 9, wherein the processor and the interactive graphical user interface generation server are configured to:
determine whether the corresponding high risk member metric of each selectable outline exceeds a fourth threshold;
in response to determining the corresponding high risk member metric of the selectable outline exceeds the fourth threshold, transform the saturation of the selectable outline into a second saturation level; and
in response to determining the corresponding high risk member metric of the selectable outline does not exceed the fourth threshold, transform the saturation of the selectable outline into a third saturation level,
wherein the first saturation level is greater than the second saturation level, and
wherein the second saturation level is greater than the third saturation level.

11. A method for automatically transforming a graphical user interface according to dynamically generated data structures, the method comprising:
retrieving a first raw data structure from a first data server;
retrieving a second raw data structure from a second data server;
generating a first processed data structure from the first raw data structure and the second raw data structure;
retrieving a third raw data structure from a storage device;
generating a second processed data structure from the third raw data structure;
generating a third processed data structure from the first processed data structure and the second processed data structure;
transforming the graphical user interface by displaying a first selectable element;
transforming the graphical user interface by displaying a second selectable element;
transforming the graphical user interface by displaying a plurality of selectable outlines, each selectable outline generated according to the third processed data structure;
in response to a user selecting the first selectable element, automatically transforming a color of each of the plurality of selectable outlines according to the third processed data structure; and
in response to the user selecting the second selectable element, automatically transforming a saturation level of each of the plurality of selectable outlines according to the third processed data structure.

12. The method of claim 11, wherein the third processed data structure comprises a plurality of metrics corresponding to a plurality of geographical regions.

13. The method of claim 12, wherein each outline of the plurality of selectable outlines corresponds to a geographical region of the plurality of geographical regions.

14. The method of claim 13, wherein:
the third processed data structure comprises a plurality of case rate metrics, and
each case rate metric is associated with a corresponding geographical region of the plurality of geographical regions.

15. The method of claim 14, further comprising:
determining whether the corresponding case rate metric of each selectable outline exceeds a first threshold; and
in response to determining the corresponding case rate metric of the selectable outline exceeds the first threshold, transforming the selectable outline into a first color.

16. The method of claim 15, further comprising:
determining whether the corresponding case rate metric of each selectable outline exceeds a second threshold; and
in response to determining the corresponding case rate metric of the selectable outline exceeds the second threshold, transforming the selectable outline into a second color.

17. The method of claim 16, further comprising:
in response to determining the corresponding case rate metric of the selectable outline does not exceed the second threshold, transforming the selectable outline into a third color.

18. The method of claim 13, wherein:
the third processed data structure comprises a plurality of percentage high risk member metrics, and
each percentage high risk member metric is associated with a corresponding region of the plurality of geographic regions.

19. The method of claim 18, further comprising:
determining whether the corresponding high risk member metric of each selectable outline exceeds a third threshold; and
in response to determining the corresponding high risk member metric of the selectable outline exceeds the third threshold, transforming a saturation of the selectable outline into a first saturation level.

20. The method of claim 19, further comprising:
determining whether the corresponding high risk member metric of each selectable outline exceeds a fourth threshold;
in response to determining the corresponding high risk member metric of the selectable outline exceeds the fourth threshold, transforming a saturation of the selectable outline into a second saturation level; and
in response to determining the corresponding high risk member metric of the selectable outline does not exceed the fourth threshold, transforming a saturation of the selectable outline into a third saturation level,
wherein the first saturation level is greater than the second saturation level, and
wherein the second saturation level is greater than the third saturation level.
